SEQUENCE LISTING (6 sequences)

SEQ ID NO: 1 - protein, 186 aa, Hepatitis C virus
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 2 - protein, 10 aa, Artificial Sequence
  HCV NS4A/4B protease cleavage site
DEMEECSQHL

SEQ ID NO: 3 - protein, 9 aa, Artificial Sequence
  HCV NS5A/5B protease cleavage site
EDVVPCSMG

SEQ ID NO: 4 - protein, 185 aa, Artificial Sequence
  phosphotyrosine binding domain
MGKPLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRDIEQVPQQPTLK

SEQ ID NO: 5 - protein, 7 aa, Artificial Sequence
  HIF1a degron
MLAPYIP

SEQ ID NO: 6 - protein, 186 aa, Artificial Sequence
  phosphotyrosine binding domain with HIF1a degron
MGKPLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGMLAPYIPEYVAYVAKDPVNQRACHILECPEGLAQDVISTIGQAFELRFKQYLRDIEQVPQQPTLK

(19) United States
(12) Patent Application Publication
(10) Pub. No.: US 2019/0024070 A1
(43) Pub. Date: Jan. 24, 2019

(54) REWIRING ABERRANT CANCER SIGNALING TO A THERAPEUTIC EFFECTOR RESPONSE WITH A SYNTHETIC TWO-COMPONENT SYSTEM

(71) Applicant: The Board of Trustees of the Leland Stanford Junior University, Stanford, CA (US)

(72) Inventors: Hokyung Chung, La Jolla, CA (US); Michael Z. Lin, Stanford, CA (US)

(21) Appl. No.: 16/044,131

(22) Filed: Jul. 24, 2018

Related U.S. Application Data
(63) Continuation of application No. 62/536,165, filed on Jul. 24, 2017.

(51) Int. Cl. C12N 9/48; A61P 35/00; C07K 14/47; C12N 9/22; C12N 15/11

(52) U.S. Cl. CPC C12N 9/48; A61P 35/00; C12Y 304/21098; C07K 14/4747; C07K 14/4702; A61K 38/00; C12N 15/11; C07K 2319/50; C07K 2319/72; C12N 2800/80; C12N 2310/20; C12N 9/22

(57) ABSTRACT

Compositions and methods for targeted treatment of cancer are disclosed. In particular, the invention relates to methods of targeting anti-cancer therapy to cells exhibiting aberrant signaling associated with cancer pathogenesis by administering synthetic signaling proteins that couple detection of an oncogenic signal to release of therapeutic agents into cancerous cells.

### CROSS-REFERENCE TO RELATED APPLICATION

This application claims benefit under 35 U.S.C. § 119(e) of provisional application 62/536,165, filed Jul. 24, 2017, which application is hereby incorporated by reference in its entirety.

### STATEMENT REGARDING FEDERALLY SPONSORED RESEARCH OR DEVELOPMENT

This invention was made with government support under contract GM098734 awarded by the National Institutes of Health. The government has certain rights in the invention.

### TECHNICAL FIELD

The present invention pertains generally to the field of cancer therapy. In particular, the invention relates to methods of targeting anti-cancer therapy to cells exhibiting aberrant signaling associated with cancer pathogenesis by administering synthetic signaling proteins that couple detection of an oncogenic signal to release of therapeutic agents into cancerous cells.

### BACKGROUND

Many cancers are driven by mutations that cause constitutive activation of signaling networks promoting cell growth, proliferation, or survival. For example, constitutive activation of ErbB-family receptor tyrosine kinases by mutation or overexpression occurs in 20-30% of solid tumors. Pharmacological approaches to cancer therapy that aim at blocking tumor-promoting signals or initiating an immune response to a cell surface marker suffer from toxicity from inhibition of normal physiological processes utilizing the same signals (FIG. 1A), and often encounter resistance due to target site mutation or compensatory second-site mutations. Pharmacological approaches to induce synthetic lethality specifically in cancer cells by blocking other protein functions are limited by the small set of known synthetic dependencies and also select for resistance.

Thus, therapies that can differentiate between normal and tumorigenic levels of signaling pathway activation, and that are not defeated by increased or maintained pathway activation, would be highly desirable.

### SUMMARY

In particular, the invention relates to methods of targeting anti-cancer therapy to cells exhibiting aberrant signaling associated with cancer pathogenesis by administering synthetic signaling proteins that couple detection of an oncogenic signal to release of therapeutic agents into cancerous cells.

In one aspect, the invention includes a method for targeted treatment of a cancer associated with hyperactivity of a receptor tyrosine kinase, the method comprising: a) administering to a subject in need thereof a therapeutically effective amount of a first fusion protein comprising a protease connected to a phosphotyrosine binding (PTB) domain capable of binding to a phosphorylated tyrosine residue on the receptor tyrosine kinase; and b) administering a therapeutically effective amount of a second fusion protein comprising an SH2 domain connected to i) a substrate comprising a cleavage site recognized by the protease and ii) an anti-cancer therapeutic agent, wherein cleavage of the substrate at the cleavage site by the protease of the first fusion protein releases the anti-cancer therapeutic agent from the second fusion protein.

In one embodiment, the receptor tyrosine kinase is a hyperactive ErbB receptor tyrosine kinase.

In another embodiment, the protease is a hepatitis C virus (HCV) NS3 protease.

In another embodiment, the PTB domain comprises the amino acid sequence of SEQ ID NO:4, or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the PTB domain is capable of binding to a phosphorylated tyrosine residue on the receptor tyrosine kinase.

In another embodiment, the first fusion protein further comprises a degron, wherein degradation activity of the degron is inhibited by binding of the PTB domain of the fusion protein to the phosphorylated tyrosine residue on the receptor tyrosine kinase such that the fusion protein accumulates preferentially in cancerous cells.

In another embodiment, the degron is an HIF1a degron comprising the amino acid sequence of SEQ ID NO:5, or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the degron is capable of promoting degradation of a fusion protein containing it.

In another embodiment, the degron is located in a loop of the PTB domain. In certain embodiments, the PTB domain with the degron inserted comprises the amino acid sequence of SEQ ID NO:6, or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the PTB domain is capable of binding to a phosphorylated tyrosine residue on the receptor tyrosine kinase, and the degron is capable of promoting degradation of a fusion protein containing it.

In another embodiment, the PTB is a Shc PTB.

In another embodiment, the SH2 domain is a Vav1 SH2 domain.

In another embodiment, the tyrosine kinase receptor is constitutively phosphorylated at the tyrosine residue.

In another embodiment, the cancer is selected from the group consisting of breast cancer, colorectal cancer, head and neck cancer, brain cancer, and lung cancer.

In another embodiment, the first fusion protein or the second fusion protein is provided by a vector (e.g., a non-viral or viral vector). For example, a non-integrating viral vector such as an adeno-associated virus may be used.

Anti-cancer therapeutic agents may include, but are not limited to, chemotherapy, immunotherapy, and biologic agents. In certain embodiments, the anti-cancer therapeutic agent is a pro-apoptotic protein (e.g., BAX) or a transcription factor that activates a pro-apoptotic gene (e.g., FoxO3).

In another embodiment, the anti-cancer therapeutic agent comprises a complex of a catalytically inactive Cas9 (dCas9) with a guide RNA for activating or repressing expression of a gene of interest.

In another embodiment, the dCas9 is fused to a transcriptional activation domain capable of activating transcription of a gene of interest. The gene of interest may be, for example, a pro-apoptotic gene or an immunostimulatory gene. In one embodiment, the transcriptional activation domain is a VP64-p65-Rta (VPR) transcriptional activation domain.

In another embodiment, multiple cycles of treatment are administered to the subject for a time period sufficient to effect at least a partial tumor response, or more preferably, a complete tumor response.

In another embodiment, the method further comprising administering one or more chemotherapeutic agents to the subject.

In another aspect, the invention includes a method of selectively treating a cancerous cell having a hyperactive ErbB receptor tyrosine kinase in a heterogenous population of cells, the method comprising: a) contacting the population of cells with an effective amount of a first fusion protein comprising a protease connected to a phosphotyrosine binding (PTB) domain that selectively binds to a phosphorylated tyrosine residue on the hyperactive ErbB receptor tyrosine kinase; and b) contacting the population of cells with an effective amount of a second fusion protein comprising an SH2 domain connected to a substrate comprising a cleavage site recognized by the protease and an anti-cancer therapeutic agent, wherein cleavage of the substrate at the cleavage site by the protease of the first fusion protein releases the therapeutic agent from the second fusion protein inside the cancerous cell having the hyperactive ErbB receptor tyrosine kinase.

In another aspect, the invention includes a kit for treating cancer, as described herein, the kit comprising: a) a first fusion protein comprising a protease connected to a phosphotyrosine binding (PTB) domain capable of binding to a phosphorylated tyrosine residue on a hyperactive receptor tyrosine kinase; and b) a second fusion protein comprising an SH2 domain connected to a substrate comprising a cleavage site recognized by the protease and an anti-cancer therapeutic agent. The kit may further comprise means for delivering the fusion proteins to a subject. Additionally, the kit may further comprise instructions for treating cancer according to the methods described herein.

The methods of the invention may be combined with any other method of treating cancer, such as, but not limited to, surgery, radiation therapy, chemotherapy, hormonal therapy, immunotherapy, or biologic therapy.

These and other embodiments of the subject invention will readily occur to those of skill in the art in view of the disclosure herein.

### BRIEF DESCRIPTION OF THE FIGURES

FIGS. 1A-1E show the concept for a molecular integrator of ErbB signaling. FIG. 1A shows pharmacological approaches to cancer therapy that aim at blocking tumor-promoting signals or initiating an immune response to a cell surface marker suffer from toxicity from inhibition of normal physiological processes utilizing the same signals.

FIG. 1B shows that signal-induced proteolysis can integrate signal activity over time and function as a generalizable activation mechanism for multiple effectors. FIG. 1C shows molecular modeling suggesting that the mKO2-substrate-CAAX protein should be able to be cleaved by ShcPTB-NS3 bound to ErbB. FIG. 1D shows observed cleavage efficiency by protease and substrate variants. Breast cancer BT-474 cells were transfected with the indicated constructs with or without 0.5 μM ErbB inhibitor lapatinib, which creates an ErbB-inactive condition as a negative control. After 24 hours, cells were lysed for immunoblotting against a v5 epitope tag fused to mKO2 and GAPDH, serving as a loading control. FIG. 1E shows quantitation of percent cleavage of substrates (n=3, error bars represent s.e.m).

FIGS. 2A-2D show that dual-targeting of protease and substrate to the receptor complex improves oncogenic ErbB signal-dependent proteolysis. FIG. 2A shows a schematic of the dual-targeted system. Substrate is recruited to the active receptor via SH2 which is expected to facilitate the substrate (line between SH2 and cargo) cleavage. FIG. 2B shows an atomic model of the dual-targeted system. FIG. 2C shows the observed cleavage efficiency by the mon- and dual-targeted system. BT474 cells expressed the indicated constructs for 24 hours and were lysed subsequently for immunoblotting against a v5 epitope tag fused to mKO2 and GAPDH, serving as a loading control. FIG. 2D shows quantitation of observed percent cleavage of the substrates (n=3, error bars represent s.e.m).

FIGS. 3A-3F show that reduction of protease stability improves the selectivity of ErbB activation-dependent proteolysis. FIG. 3A shows a schematic of protease stability regulation upon phosphorylated receptor binding. FIG. 3B shows a structural model of the PTBhif-NS3. Hif-1a degron (pink) is inserted in the loop near the phosphorylated peptide binding site. FIG. 3C shows the half-life measurement of PTB-NS3 and PTBhif-NS3 in the presence or the absence of the lapatinib, using the SMASh technique (n=3, error bars represent s.e.m.). Values were fit to a monoexponential decay curve to calculate half-lives. FIG. 3D shows the actual ErbB-dependent mKO2 release. BT-474 cells expressed the indicated constructs for 24 hours and were lysed subsequently for immunoblotting against a v5 epitope tag fused to mKO2 and GAPDH, serving as a loading control. FIG. 3E shows quantitation of the observed percent cleavage of the substrates (n=3, error bars represent s.e.m). PTBhif-NS3 and cargo-DEMEEC-SH2-CAAX were designated as the ErbB-RASER system. FIG. 3F shows verification of PTB dependence in ErbB-RASER.

FIGS. 4A-4F show characterization of the RASER system. FIG. 4A shows generalization of RASER to multiple ErbB+ cancer cells. The RASER system shows substrate release in ErbB over-activated cancer cell lines such as BT-474 and SK-BR-3 (human breast cancer), 4T1 (mouse breast cancer), SK-OV-3 (human ovarian cancer) and LN299 EGFRvIII (human glioblastoma). Substrate release was blocked by the ErbB inhibitor lapatinib. FIG. 4B shows the generalizability and selectivity of the RASER system is confirmed with fluorescence microscopy. scale bar, 20 μm. FIG. 4C shows that RASER is specific for constitutively active ErbB, rather than ErbB activated by physiological levels of EGF. MCF7 (which express normal ErbB level), SK-BR-3 and BT-474 (aberrant ErbB2 level) cells were transfected with the RASER construct. After 16 hours of protein expression, MCF7 cells were stimulated by 50 nM of EGF for 1 hour to 16 hours as indicated to recapitulate the temporal activation of ErbB. After 32 hours of protein expression, cells were lysed for immunoblotting to detect against phosphorylated ErbBs, mKO2 and GAPDH. FIG. 4D shows quantitation of mKO2 immunoblot signals normalized to GAPDH levels (n=3, error bars represent s.e.m). FIG. 4E shows that RASER output is comparable to the natural downstream effect of the active ErbB. Phospho-ErbB2 and downstream of ErbB, phosphorylated Akt and phosphorylated Erk as well as released mKO2 were detected by western. FIG. 4F shows quantitation of fold induction of Akt, Erk, and RASER (mKO2) between lapatinib treated (ErbB off) and untreated (ErbB on) cells (n=3, error bars represent s.e.m.).

FIGS. 5A-5C show that RASER can be programmed to induce apoptosis in cancer cells. FIG. 5A shows a schematic description of the ErbB-RASER-Bax system. Bax monomer is released in the presence of tumorigenic ErbB signaling activation. FIG. 5B shows results for MCF7 cells (with normal ErbB levels) and BT-474 cells (which overexpress ErbB2) transfected with the ErbB-RASER-Bax construct. After 16 hours of protein expression, cells were lysed for immunoblotting to detect BAX, cleaved PARP and GAPDH. FIG. 5C shows quantitation of cleaved PARP levels in immunoblots of RASER-transfected cells compared to mock-transfected cells (n=3, error bars represent s.e.m.).

FIGS. 6A-6C show that RASER can be programmed to induce transcription of endogenous genes in cancer cells. FIG. 6A shows a schematic description of the ErbB-RASER-FoxO3 system. Constitutively active FoxO3 (FoxO3-QM) is released in the presence of tumorigenic ErbB signaling activation. The released FoxO3-QM activates pro-apoptotic target genes including Bim. FIG. 6B shows results for MCF7 cells (with normal ErbB levels) and BT-474 cells (which overexpress ErbB2) transfected with the ErbB-RASER-FoxO construct. After 16 hours of protein expression, cells were lysed for immunoblotting to detect FoxO3-QM, cleaved PARP, and GAPDH. FIG. 6C shows quantitation of cleaved PARP levels in immunoblots of RASER-transfected cells compared to mock-transfected cells (n=3, error bars represent s.e.m.).

FIGS. 7A-7C show that RASER can be programmed to induce transcription of target genes via dCas9. FIG. 7A shows a schematic of the RASER system for selective transcription with VPRdCas9. FIG. 7B shows results with a plasmid expressing VPRdCas9-substrate-SH2-CAAX or VPRdCas9 or no protein cotransfected with a multi-cistronic plasmid expressing sgRNA, PTBhifNS3, and mClover3 GFP into BT-474 with or without lapatinib. Cells were imaged 24 hours after transfection. FIG. 7C shows quantification of mCherry fluorescence showing that transcriptional activation by ErbB-RASER-VPRCas9 is as efficient as the VPRCas9 positive control and is ErbB-dependent. The mCherry fluorescence was measured in GFP+ cells cotransfected with VPRdCas9-substrate-SH2-CAAX or VPRdCas9 and the multi-cistronic plasmid, after subtraction of mCherry levels in cells cotransfected with the multi-cistronic plasmid alone (n=10). Error bars are SEM.

### DETAILED DESCRIPTION

The practice of the present invention will employ, unless otherwise indicated, conventional methods of medicine, pharmacology, chemistry, biochemistry, molecular biology and recombinant DNA techniques and immunology, within the skill of the art. Such techniques are explained fully in the literature. See, e.g., R. A. Weinberg The Biology of Cancer (Garland Science, 2^{nd} edition, 2013); Apoptosis in Cancer Pathogenesis and Anti-cancer Therapy: New Perspectives and Opportunities (Advances in Experimental Medicine and Biology, C. D. Gregory ed., Springer, 2016); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a cell” includes a mixture of two or more cells, and the like.

The term “about,” particularly in reference to a given quantity, is meant to encompass deviations of plus or minus five percent.

The terms “fusion protein” or “fusion polypeptide,” as used herein refer to a fusion comprising a protease in combination with a PTB domain or a fusion comprising an SH2 domain in combination with a substrate for the protease and an anti-cancer therapeutic agent as part of a single continuous chain of amino acids, which chain does not occur in nature. The fusion protein comprising the protease in combination with the PTB domain may further comprise a degron, wherein degradation activity of the degron is inhibited by binding of the PTB domain to a phosphorylated tyrosine residue on a receptor tyrosine kinase such that the fusion protein accumulates preferentially in cancerous cells. The fusion polypeptides may also contain additional sequences, such as targeting or localization sequences, detectable labels, or tag sequences.

The term “cleavage site” refers to the bond (e.g. a scissile bond) cleaved by an agent. A cleavage site for a protease includes the specific amino acid sequence recognized by the protease during proteolytic cleavage and typically includes the surrounding one to six amino acids on either side of the scissile bond, which bind to the active site of the protease and are needed for recognition as a substrate.

As used herein, a “degron” is an amino acid sequence that targets a protein for cellular degradation and specifies degradation of itself and any fusion protein of which it is a part. The degron may promote degradation of an attached polypeptide, for example, through either the proteasome or autophagy-lysosome pathways.

The terms “polypeptide” and “protein” refer to a polymer of amino acid residues and are not limited to a minimum length. Thus, peptides, oligopeptides, dimers, multimers, and the like, are included within the definition. Both full length proteins and fragments thereof are encompassed by the definition. The terms also include post-expression modifications of the polypeptide, for example, glycosylation, acetylation, phosphorylation, hydroxylation, and the like. Furthermore, for purposes of the present invention, a “polypeptide” refers to a protein which includes modifications, such as deletions, additions and substitutions to the native sequence, so long as the protein maintains the desired activity. These modifications may be deliberate, as through site directed mutagenesis, or may be accidental, such as through mutations of hosts which produce the proteins or errors due to PCR amplification.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

By “fragment” is intended a molecule consisting of only a part of the intact full-length sequence and structure. The fragment can include a C-terminal deletion an N-terminal deletion, and/or an internal deletion of the polypeptide. Active fragments of a particular protein or polypeptide will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence, provided that the fragment in question retains biological activity, such as catalytic activity, ligand binding activity, regulatory activity, degron protein degradation signaling, or fluorescence characteristics.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. These terms include, but are not limited to, breast cancer, colorectal cancer, head and neck cancer, brain cancer, prostate cancer, lung cancer, ovarian cancer, testicular cancer, colon cancer, pancreatic cancer, gastric cancer, hepatic cancer, leukemia, lymphoma, adrenal cancer, thyroid cancer, pituitary cancer, renal cancer, and skin cancer.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

By “therapeutically effective dose or amount” of each of the first and second fusion proteins is intended an amount that when administered in combination brings about a positive therapeutic response with respect to treatment of an individual for cancer. Of particular interest is an amount of the fusion proteins that provides anti-tumor activity, as defined herein. By “positive therapeutic response” is intended the individual undergoing treatment according to the invention exhibits an improvement in one or more symptoms of the cancer for which the individual is undergoing therapy. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA or RNA moieties into suitable host cells. The term refers to both stable and transient uptake of the genetic material, and includes uptake, for example, of recombinant nucleic acids encoding fusion proteins.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Operably linked” refers to an arrangement of elements wherein the components so described are configured so as to perform their usual function. For example, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence. In another example, a degron operably linked to a polypeptide is capable of promoting degradation of the polypeptide when the proper cellular degradation system (e.g., proteasome or autophagosome degradation) is present. The degron need not be contiguous with the polypeptide, so long as it functions to direct degradation of the polypeptide.

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant” refers to biologically active derivatives of the reference molecule that retain desired activity, such as RNA interference (RNAi), lncRNA inhibition, or transcription factor inhibition. In general, the term “variant” refers to molecules having a native sequence and structure with one or more additions, substitutions (generally conservative in nature) and/or deletions, relative to the native molecule, so long as the modifications do not destroy biological activity and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The terms “subject” refers to a vertebrate subject, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; and birds, including domestic, wild and game birds such as chickens, turkeys and other gallinaceous birds, ducks, geese, and the like. The term does not denote a particular age. Thus, both adult and newborn individuals are intended to be covered.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the development of a method for targeting anti-cancer therapy to cells exhibiting aberrant signaling associated with cancer pathogenesis. The general method utilizes oncogenic signal-induced proteolysis to release tethered therapeutic agents inside cancerous cells, an approach referred to as rewiring of aberrant signaling to effector release (RASER). The inventors have engineered a compact two-component system to sense constitutive ErbB phosphorylation and trigger therapeutic responses (Example 1). Modular sensing and actuation domains in this system allow facile optimization of the sensing and versatile programming of therapeutic outputs. The resulting system, responds specifically to constitutively active ErbB, and can be programmed to induce a variety of outputs including direct induction of apoptosis and transcription of apoptosis-inducing genes. The RASER system is generalizable to various cancers by customizing sensor-actuator modules to specific oncogenic signals.

In order to further an understanding of the invention, a more detailed discussion is provided below regarding RASER systems and methods of using such systems to treat cancer.

A. RASER Systems

In one embodiment, the RASER system is designed for targeted treatment of a cancer comprising a hyperactive receptor tyrosine kinase. A two-component system is used comprising two fusion proteins: i) a first fusion protein comprising a protease connected to a phosphotyrosine binding (PTB) domain capable of binding to a phosphorylated tyrosine residue on a hyperactive receptor tyrosine kinase in a cancerous cell; and ii) a second fusion protein comprising an SH2 domain connected to a substrate comprising a cleavage site recognized by the protease and an anti-cancer therapeutic agent. Cleavage of the substrate by the protease of the first fusion protein releases the therapeutic agent from the second fusion protein inside a cancerous cell.

Exemplary proteases which can be used in the first fusion protein include hepatitis C virus proteases (e.g., NS3 and NS2-3); signal peptidase; proprotein convertases of the subtilisin/kexin family (furin, PC1, PC2, PC4, PACE4, PCS, PC); proprotein convertases cleaving at hydrophobic residues (e.g., Leu, Phe, Val, or Met); proprotein convertases cleaving at small amino acid residues such as Ala or Thr; proopiomelanocortin converting enzyme (PCE); chromaffin granule aspartic protease (CGAP); prohormone thiol protease; carboxypeptidases (e.g., carboxypeptidase E/H, carboxypeptidase D and carboxypeptidase Z); aminopeptidases (e.g., arginine aminopeptidase, lysine aminopeptidase, aminopeptidase B); prolyl endopeptidase; aminopeptidase N; insulin degrading enzyme; calpain; high molecular weight protease; and, caspases 1, 2, 3, 4, 5, 6, 7, 8, and 9. Other proteases include, but are not limited to, aminopeptidase N; puromycin sensitive aminopeptidase; angiotensin converting enzyme; pyroglutamyl peptidase II; dipeptidyl peptidase IV; N-arginine dibasic convertase; endopeptidase 24.15; endopeptidase 24.16; amyloid precursor protein secretases alpha, beta and gamma; angiotensin converting enzyme secretase; TGF alpha secretase; TNF alpha secretase; FAS ligand secretase; TNF receptor-I and -II secretases; CD30 secretase; KL1 and KL2 secretases; IL6 receptor secretase; CD43, CD44 secretase; CD16-I and CD16-II secretases; L-selectin secretase; Folate receptor secretase; MMP 1, 2, 3, 7, 8, 9, 10, 11, 12, 13, 14, and 15; urokinase plasminogen activator; tissue plasminogen activator; plasmin; thrombin; BMP-1 (procollagen C-peptidase); ADAM 1, 2, 3, 4, 5, 6, 7, 8, 9, 10, and 11; and, granzymes A, B, C, D, E, F, G, and H. The protease chosen for use in the fusion protein is preferably highly selective for the cleavage site in the cleavable linker. Additionally, protease activity is preferably inhibitable with inhibitors that are cell-permeable and not toxic to the cell or subject under study. For a discussion of proteases, see, e.g., V. Y. H. Hook, Proteolytic and cellular mechanisms in prohormone and proprotein processing, RG Landes Company, Austin, Tex., USA (1998); N. M. Hooper et al., Biochem. J. 321: 265-279 (1997); Z. Werb, Cell 91: 439-442 (1997); T. G. Wolfsberg et al., J. Cell Biol. 131: 275-278 (1995); K. Murakami and J. D. Etlinger, Biochem. Biophys. Res. Comm. 146: 1249-1259 (1987); T. Berg et al., Biochem. J. 307: 313-326 (1995); M. J. Smyth and J. A. Trapani, Immunology Today 16: 202-206 (1995); R. V. Talanian et al., J. Biol. Chem. 272: 9677-9682 (1997); and N. A. Thornberry et al., J. Biol. Chem. 272: 17907-17911 (1997), the disclosures of which are incorporated herein.

In certain embodiments, the protease used in the first fusion protein is a hepatitis C virus (HCV) nonstructural protein 3 (NS3) protease. NS3 consists of an N-terminal serine protease domain and a C-terminal helicase domain. The protease domain of NS3 forms a heterodimer with the HCV nonstructural protein 4A (NS4A), which activates proteolytic activity. An NS3 protease may comprise the entire NS3 protein or a proteolytically active fragment thereof and may further comprise an activating NS4A region.

The cleavage site in the second fusion protein is designed for selective cleavage by the particular protease included in the first fusion protein. The cleavage site includes the specific amino acid sequence recognized by the protease during proteolytic cleavage and typically includes the surrounding one to six amino acids on either side of the scissile bond, which bind to the active site of the protease and are needed for recognition as a substrate. The substrate for the protease in the second fusion protein may contain any protease recognition motif known in the art and is typically cleavable under physiological conditions.

In certain embodiments, an NS3 protease is used in the first fusion protein and a corresponding NS3 cleavage site in the second fusion protein. NS3 nucleic acid and protein sequences may be derived from HCV, including any isolate of HCV having any genotype (e.g., seven genotypes 1-7) or subtype. A number of NS3 nucleic acid and protein sequences are known. A representative NS3 sequence is presented in SEQ ID NO:1. Additional representative sequences are listed in the National Center for Biotechnology Information (NCBI) database. See, for example, NCBI entries: Accession Nos. YP_001491553, YP_001469631, YP_001469632, NP_803144, NP_671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, AB136969, ABN05226, KF516075, KF516074, KF516056, AB826684, AB826683, JX171009, JX171008, JX171000, EU847455, EF154714, GU085487, JX171065, JX171063, all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct a fusion protein or a recombinant polynucleotide encoding such a fusion protein, as described herein. In one embodiment, a slower-cleaving T54A mutant of NS3 protease is used in the first fusion protein (numbering is relative to the reference sequence of SEQ ID NO:1, and it is to be understood that the corresponding positions in NS3 proteases obtained from other HCV strains are also intended to be encompassed by the present invention).

Exemplary NS3 protease cleavage sites, which can be used in the substrate of the second fusion protein, include the four junctions between nonstructural (NS) proteins of the HCV polyprotein normally cleaved by the NS3 protease during HCV infection, including the NS3/NS4A, NS4A/NS4B, NS4B/NS5A, and NS5A/NS5B junction cleavage sites. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; herein incorporated by reference in its entirety.

The second fusion protein also carries a cargo comprising an anti-cancer therapeutic agent, which is released inside cells upon proteolytic cleavage of the second fusion protein by the protease of the first fusion protein. Exemplary anti-cancer therapeutic agents include chemotherapy, immunotherapy, and biologic agents.

For example, chemotherapy agents include, but are not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Biologic anti-cancer therapeutic agents include, but are not limited to, small molecule inhibitors or monoclonal antibodies such as, but not limited to, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy).

Immunotherapy anti-cancer therapeutic agents include, but are not limited to, cancer vaccines (e.g., Hepcortespenlisimut-L, Sipuleucel-T), anti-cancer therapeutic antibodies (e.g., Alemtuzumab, Ipilimumab, Ofatumumab, Nivolumab, Pembrolizumab, or Rituximab), cytokines (e.g., interferons, including type I (IFNα and IFNβ), type II (IFNγ) and type III (IFNλ) and interleukins, including interleukin-2 (IL-2)), adjuvants (e.g., polysaccharide-K), and immune checkpoint blockade therapeutic agents.

In some embodiments, the anti-cancer therapeutic agent comprises a pro-apoptotic protein or tumor suppressor, such as, but not limited to, BAX, BID, BAK, BAD, apoptotic protease activating factor 1 (APAF1), p53, p73, pVHL, APC, CD95, STS, YPEL3, ST7, and ST14. In other embodiments, the anti-cancer therapeutic agent comprises a transcription factor that activates pro-apoptotic genes, such as, but not limited to, Forkhead box O (FOXO) transcription factors (e.g., FoxO3), AP-2 alpha, activating transcription factor 5 (ATFS), C/EBP homologous protein (CHOP), and E2F1.

In yet another embodiment, the anti-cancer therapeutic agent comprises a complex of a catalytically inactive Cas9 (dCas9) with a guide RNA for activating or repressing expression of a gene of interest. An engineered nuclease-deactivated Cas9 (dCas9) is used to allow sequence-specific targeting without cleavage. Nuclease-deactivated forms of Cas9 may be engineered by mutating catalytic residues at the active site of Cas9 to destroy nuclease activity. Any such nuclease deficient Cas9 protein from any species may be used as long as the engineered dCas9 retains sgRNA-mediated sequence-specific targeting. In particular, the nuclease activity of Cas9 from Streptococcus pyogenes can be deactivated by introducing two mutations (D10A and H841A) in the RuvC1 and HNH nuclease domains. Other engineered dCas9 proteins may be produced by similarly mutating the corresponding residues in other bacterial Cas9 isoforms. For a description of engineered nuclease-deactivated forms of Cas9, see, e.g., Qi et al. (2013) Cell 152:1173-1183, Dominguez et al. (2016) Nat. Rev. Mol. Cell. Biol. 17(1):5-15; herein incorporated by reference in their entireties.

A nuclease-deactivated Cas9 protein can be designed to target particular nucleic acid sequences by altering its guide RNA sequence. A target-specific single guide RNA (sgRNA) comprises a nucleotide sequence that is complementary to a target site, and thereby mediates binding of the dCas9-sgRNA complex by hybridization at the target site. The sgRNA can be designed, for example, with a sequence complementary to a gene regulatory or exonic sequence. The target site will typically comprise a nucleotide sequence that is complementary to the sgRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide.

In certain embodiments, the sgRNA comprises 5-50 nucleotides, 10-30 nucleotides, 15-25 nucleotides, 18-22 nucleotides, 19-21 nucleotides, and any length between the stated ranges, including, for example, 10, 11, 12, 13, 14, 15, 16, 17, 18, 19, 20, 21, 22, 23, 24, 25, 26, 27, 28, 29, or 30 nucleotides.

The sgRNAs are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109.

In some embodiments, the dCas9 is fused to a transcriptional activation domain capable of activating transcription of a gene of interest such as a pro-apoptotic gene or an immunostimulatory gene. In one embodiment, the transcriptional activation domain is a VP64-p65-Rta (VPR) transcriptional activation domain.

In certain embodiments, the first fusion protein further comprises a degron to allow control of the release of the anti-cancer therapeutic agent so as to avoid release inside normal noncancerous cells, but allow release in cancerous cells. The degron provides a degradation signal that targets the fusion protein for cellular degradation through either the proteasome or autophagy-lysosome pathway. In the first fusion protein, the degron is operably linked to the protease such that degradation of the protease prevents cleavage and release of the anti-cancer therapeutic agent from the second fusion protein in normal or noncancerous cells. The degron must be operably linked to the protease, but need not be contiguous with it as long as the degron still functions to direct degradation of the protease. Preferably, the degron induces rapid degradation of the fusion protein, including the protease in noncancerous cells.

The first fusion protein is designed such that the degradation activity of the degron is controllable. For example, the degron can be inserted in a loop of the PTB domain such that degron activity is inhibited by binding of the PTB domain to a phosphorylated tyrosine residue of a receptor tyrosine kinase in a cancerous cell. Fusion proteins with degrons so inhibited are not degraded; hence, the fusion protein with its attached active protease accumulates preferentially in cancerous cells. Cleavage of the anti-cancer therapeutic agent from the second fusion protein releases the anti-cancer therapeutic agent inside the cancerous cell.

Any suitable degron may be used, including, but not limited to, N-degrons of type 1 (e.g., degron sequence comprises positively charged amino acids such as Arg, Lys, and His) or type 2 (degron sequences comprises bulky hydrophobic amino acids such as Phe, Trp, Tyr, Leu, and Ile), phosphodegrons (e.g., Cdc4 or Fbw7 degron), or oxygen-dependent degrons (e.g., a hypoxia-inducible factor alpha (HIF-a) degron). Engineered small-molecule-dependent, inducible degrons (e.g. engineered auxin-inducible degrons) may also be used (see, e.g., Nishimura et al. (2009) Nat. Methods 6(12):917-922). Degrons may further comprise post-translational modifications, including phosphorylation and hydroxylation. For a discussion of degrons and their function in protein degradation, see, e.g., Guharoy et al. (2016) Nat. Commun. 7:10239, Lucas et al. (2017) Curr. Opin. Struct. Biol. 44:101-110, Kanemaki et al. (2013) Pflugers Arch. 465(3):419-425, Erales et al. (2014) Biochim Biophys Acta 1843(1):216-221, Schrader et al. (2009) Nat. Chem. Biol. 5(11):815-822, Ravid et al. (2008) Nat. Rev. Mol. Cell. Biol. 9(9):679-690, Tasaki et al. (2007) Trends Biochem Sci. 32(11):520-528, Meinnel et al. (2006) Biol. Chem. 387(7):839-851, Kim et al. (2013) Autophagy 9(7):1100-1103, Varshaysky (2012) Methods Mol. Biol. 832:1-11, and Fayadat et al. (2003) Mol. Biol. Cell. 14(3):1268-1278; herein incorporated by reference.

The polypeptides included in the fusion constructs may be connected directly to each other by peptide bonds or may be separated by intervening amino acid sequences (i.e., linkers). The fusion polypeptides may also contain additional sequences, such as tag sequences or detectable labels to facilitate cloning, purification, or detection.

Linker amino acid sequences are typically short, e.g., 20 or fewer amino acids (i.e., 20, 19, 18, 17, 16, 15, 14, 13, 12, 11, 10, 9, 8, 7, 6, 5, 4, 3, 2, or 1). Examples include short peptide sequences which facilitate cloning, poly-glycine linkers (Glyₙ where n=2, 3, 4, 5, 6, 7, 8, 9, 10 or more), histidine tags (Hisₙ where n=3, 4, 5, 6, 7, 8, 9, 10 or more), linkers composed of glycine and serine residues or glycine, serine, and alanine residues, wherein n=1, 2, 3, 4, 5, 6, 7, 8, 9, 10, 11, 12, 13, 14, 15 or more), GSAT, SEG, and Z-EGFR linkers. Linkers may include restriction sites, which aid cloning and manipulation. Other suitable linker amino acid sequences will be apparent to those skilled in the art. (See e.g., Argos (1990) J. Mol. Biol. 211(4):943-958; Crasto et al. (2000) Protein Eng. 13:309-312; George et al. (2002) Protein Eng. 15:871-879; Arai et al. (2001) Protein Eng. 14:529-532; and the Registry of Standard Biological Parts (partsregistry.org/Protein_domains/Linker).

In certain embodiments, tag sequences are located at the N-terminus or C-terminus of a fusion protein. Exemplary tags that can be used in the practice of the invention include a His-tag, a Strep-tag, a TAP-tag, an S-tag, an SBP-tag, an Arg-tag, a calmodulin-binding peptide tag, a cellulose-binding domain tag, a DsbA tag, a c-myc tag, a glutathione S-transferase tag, a FLAG tag, a HAT-tag, a maltose-binding protein tag, a NusA tag, and a thioredoxin tag.

In certain embodiments, a fusion protein further comprises a detectable label. The detectable label may comprise any molecule capable of detection. Detectable labels that may be used in the practice of the invention include, but are not limited to, radioactive isotopes, stable (non-radioactive) heavy isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. Particular examples of labels that may be used with the invention include, but are not limited to radiolabels (e.g., ³H, ¹²⁵I, ³⁵S, or ³²P), stable (non-radioactive) heavy isotopes (e.g., ¹³C or ¹⁵N), phycoerythrin, Alexa dyes, fluorescein, 7-nitrobenzo-2-oxa-1,3-diazole (NBD), YPet, CyPet, Cascade blue, allophycocyanin, Cy3, Cy5, Cy7, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenical acetyl transferase, and urease. Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

B. Production of Fusion Proteins

Fusion proteins can be prepared in any suitable manner (e.g., recombinant expression, purification from cell culture, chemical synthesis, etc.). Fusion proteins may include naturally-occurring polypeptides, recombinantly produced polypeptides, synthetically produced polypeptides, or polypeptides produced by a combination of these methods. Means for preparing fusion proteins are well understood in the art. Fusion proteins are preferably prepared in substantially pure form (i.e. substantially free from other host cell or non-host cell proteins).

In one embodiment, the fusion proteins are generated using recombinant techniques. One of skill in the art can readily determine nucleotide sequences that encode the desired polypeptides using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides useful in the claimed fusion proteins that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the fusion proteins described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia-based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides and heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences. The control sequences and other regulatory sequences may be ligated to the coding sequence prior to insertion into a vector. Alternatively, the coding sequence can be cloned directly into an expression vector that already contains the control sequences and an appropriate restriction site.

In some cases, it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frupperda, and Trichoplusia ni.

Depending on the expression system and host selected, the fusion proteins of the present invention are produced by growing host cells transformed by an expression vector described above under conditions whereby the protein of interest is expressed. The selection of the appropriate growth conditions is within the skill of the art.

In one embodiment, the transformed cells secrete the fusion protein product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted fusion protein product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant fusion proteins substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced fusion proteins are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular fusion proteins of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the fusion proteins can be further purified using conventional techniques well known in the art, such as by any of the techniques described above.

Fusion proteins can also be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final peptide or polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides, including fusion proteins.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethyl styrene copolymers, divinylbenzene-chloromethyl styrene copolymers and divinylbenzene-benzhydrylaminopolystyrene copolymers.

Fusion proteins can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

C. Nucleic Acids Encoding Fusion Proteins

Nucleic acids encoding the first and second fusion proteins can be used to treat cancer. Nucleic acids described herein can be inserted into an expression vector to create an expression cassette capable of producing the fusion proteins in a suitable host cell. The first fusion protein and the second fusion protein may be provided by a single vector or separate vectors. The ability of constructs to produce the fusion proteins can be empirically determined (e.g., see Example 1 describing detection using a reporter plasmid that expresses mCherry).

Expression cassettes typically include control elements operably linked to the coding sequence, which allow for the expression of the gene in vivo in the subject species. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Enhancer elements may also be used herein to increase expression levels of the mammalian constructs. Examples include the SV40 early gene enhancer, as described in Dijkema et al., EMPO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Once complete, the constructs encoding the first and second fusion proteins can be administered to a subject using standard gene delivery protocols. Methods for gene delivery are known in the art. See, e.g., U.S. Pat. Nos. 5,399,346, 5,580,859, 5,589,466. Genes can be delivered either directly to a vertebrate subject or, alternatively, delivered ex vivo, to cells derived from the subject and the cells reimplanted in the subject.

A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference).

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr Pharm Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides of the present invention is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules encoding the first and second fusion proteins include those derived from the pox family of viruses, including vaccinia virus and avian poxvirus. By way of example, vaccinia virus recombinants expressing the first and second fusion proteins can be constructed as follows. The DNA encoding the particular fusion protein coding sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the coding sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with. respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the coding sequences of interest (for example, a fusion protein expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The synthetic expression cassette of interest can also be delivered without a viral vector. For example, the synthetic expression cassette can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991.) 1097:1-17; Straubinger et al., in Methods of Enzymology (1983), Vol. 101, pp. 512-527.

Liposomal preparations for use in the present invention include cationic (positively charged), anionic (negatively charged) and neutral preparations, with cationic liposomes particularly preferred. Cationic liposomes have been shown to mediate intracellular delivery of plasmid DNA (Feigner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. (See, also, Feigner et al., Proc. Natl. Acad. Sci. USA (1987) 84:7413-7416). Other commercially available lipids include (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes.

Similarly, anionic and neutral liposomes are readily available, such as, from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA and/or peptide(s) can also be delivered in cochleate lipid compositions similar to those described by Papahadjopoulos et al., Biochem. Biophys. Acta. (1975) 394:483-491. See, also, U.S. Pat. Nos. 4,663,161 and 4,871,488.

The expression cassette of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J. P., et al., J Microencapsul. 14(2):197-210, 1997; O'Hagan D. T., et al., Vaccine 11(2):149-54, 1993.

Furthermore, other particulate systems and polymers can be used for the in vivo or ex vivo delivery of the nucleic acid of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Felgner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998, herein incorporated by reference) may also be used for delivery of a construct of the present invention.

Additionally, biolistic delivery systems employing particulate carriers such as gold and tungsten, are especially useful for delivering synthetic expression cassettes of the present invention. The particles are coated with the synthetic expression cassette(s) to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744. Also, needle-less injection systems can be used (Davis, H. L., et al, Vaccine 12:1503-1509, 1994; Bioject, Inc., Portland, Oreg.).

Recombinant vectors carrying a synthetic expression cassette of the present invention are formulated into compositions for delivery to a vertebrate subject (e.g., mammalian subject, preferably human). These compositions may either be prophylactic (to prevent cancer progression) or therapeutic (to treat cancer). The compositions will comprise a “therapeutically effective amount” of the nucleic acid of interest such that amounts of the first and second fusion proteins can be produced in vivo sufficient to have anti-cancer activity in the individual to which it is administered. The exact amounts necessary will vary depending on the subject being treated; the age and general condition of the subject to be treated; the degree of protection desired; the severity of the condition being treated; the particular anti-cancer therapeutic agent released in cancerous cells by the fusion proteins, and the mode of administration, among other factors. An appropriate effective amount can be readily determined by one of skill in the art. Thus, a “therapeutically effective amount” will fall in a relatively broad range that can be determined through routine trials.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of nucleic acid uptake and/or expression can also be included in the compositions or coadministered.

Once formulated, the compositions of the invention can be administered directly to the subject (e.g., as described above) or, alternatively, delivered ex vivo, to cells derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

Direct delivery of synthetic expression cassette compositions in vivo will generally be accomplished with or without viral vectors, as described above, by injection using either a conventional syringe, needless devices such as Bioject or a gene gun, such as the Accell gene delivery system (PowderMed Ltd, Oxford, England).

D. Pharmaceutical Compositions

A first fusion protein (i.e., comprising a protease connected to a phosphotyrosine binding (PTB) domain capable of binding to a phosphorylated tyrosine residue on a hyperactive receptor tyrosine kinase) and a second fusion protein (i.e., comprising an SH2 domain connected to a substrate comprising a cleavage site recognized by the protease and an anti-cancer therapeutic agent), or nucleic acids encoding them can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the fusion proteins, or nucleic acids encoding them, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the fusion proteins (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition.

Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising the first and second fusion proteins described herein are in unit dosage form, meaning an amount of a conjugate or composition of the invention appropriate for a single dose, in a premeasured or pre-packaged form.

The compositions herein may optionally include one or more additional agents, such as other drugs for treating cancer, or other medications used to treat a subject for a condition or disease. Compounded preparations may include the first and second fusion proteins and optionally, one or more drugs for treating cancer, such as one or more chemotherapeutic agents, including, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon. Alternatively, each fusion protein and/or other agents can be contained in separate compositions. The other agents may be co-administered concurrently, before, or after the fusion proteins.

C. Administration

At least one therapeutically effective dose of a first fusion protein (i.e., comprising a protease connected to a phosphotyrosine binding (PTB) domain capable of binding to a phosphorylated tyrosine residue on a hyperactive receptor tyrosine kinase) will be administered in combination with a second fusion protein (i.e., comprising an SH2 domain connected to a substrate comprising a cleavage site recognized by the protease and an anti-cancer therapeutic agent).

By “therapeutically effective dose or amount” of each of the first and second fusion proteins is intended an amount that when administered in combination brings about a positive therapeutic response with respect to treatment of an individual for cancer. Of particular interest is an amount of these agents that provides an anti-tumor effect, as defined herein. By “positive therapeutic response” is intended the individual undergoing treatment according to the invention exhibits an improvement in one or more symptoms of the cancer for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements.

In certain embodiments, one or more chemotherapeutic agents may also be administered, including, but are not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case. Generally, a therapeutically effective amount will range from about 0.50 mg to 5 grams NSAID daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID.

In certain embodiments, multiple therapeutically effective doses of each of the first and second fusion proteins and, optionally, one or more chemotherapeutic agents will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, the first and second fusion proteins and, optionally, one or more chemotherapeutic agents will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

In some embodiments, the first and second fusion proteins are administered prior to, concurrent with, or subsequent to at least one chemotherapeutic agent. If provided at the same time as the chemotherapeutic agent, the first and second fusion proteins can be provided in the same or in a different composition. Thus, the agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising the first and second fusion proteins and at least one therapeutically effective dose of a pharmaceutical composition comprising at least one chemotherapeutic agent according to a particular dosing regimen. Administration of the separate pharmaceutical compositions can be at the same time (i.e., simultaneously) or at different times (i.e., sequentially, in either order, on the same day, or on different days), so long as the therapeutic effect of the combination of these substances is caused in the subject undergoing therapy.

In other embodiments of the invention, the pharmaceutical composition comprising the agents, such as the first and second fusion proteins and/or chemotherapeutic agents, is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions comprising the first and second fusion proteins or chemotherapeutic agents may be administered using the same or different routes of administration in accordance with any medically acceptable method known in the art. Suitable routes of administration include parenteral administration, such as subcutaneous (SC), intraperitoneal (IP), intramuscular (IM), intravenous (IV), or infusion, oral and pulmonary, nasal, topical, transdermal, and suppositories. Where the composition is administered via pulmonary delivery, the therapeutically effective dose is adjusted such that the soluble level of the agent, such as the fusion proteins in the bloodstream, is equivalent to that obtained with a therapeutically effective dose that is administered parenterally, for example SC, IP, IM, or IV. In some embodiments of the invention, the pharmaceutical composition comprising the first and second fusion proteins are administered by IM or SC injection, particularly by IM or SC injection locally to the region where other therapeutic agent or agents used in cancer therapy are administered.

Factors influencing the respective amount of the various compositions to be administered include, but are not limited to, the mode of administration, the frequency of administration (i.e., daily, or intermittent administration, such as twice- or thrice-weekly), the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy.

Where a subject undergoing therapy in accordance with the previously mentioned dosing regimens exhibits a partial response, or a relapse following a prolonged period of remission, subsequent courses of therapy may be needed to achieve complete remission of the disease. Thus, subsequent to a period of time off from a first treatment period, a subject may receive one or more additional treatment periods with the first and second fusion proteins. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

D. Kits

The invention also provides kits comprising one or more containers holding compositions comprising a first fusion protein (i.e., comprising a protease connected to a phosphotyrosine binding (PTB) domain capable of binding to a phosphorylated tyrosine residue on a hyperactive receptor tyrosine kinase) and a second fusion protein (i.e., comprising an SH2 domain connected to a substrate comprising a cleavage site recognized by the protease and an anti-cancer therapeutic agent), or recombinant nucleic acids encoding them, and optionally one or more other drugs for treating cancer.

Compositions can be in liquid form or can be lyophilized, as can individual fusion proteins or nucleic acids. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for treating cancer with the fusion proteins, as described herein. The package insert can be an unapproved draft package insert or can be a package insert approved by the Food and Drug Administration (FDA) or other regulatory body.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Rewiring Aberrant Cancer Signaling to Therapeutic Effector Response with a Synthetic Two-Component System

### Introduction

Instead of attempting to block oncogenic signaling or protein function, we considered a novel approach to cancer therapy where signals driving oncogenesis are instead co-opted to trigger therapeutic responses via rewiring by synthetic signal transduction pathways. Our concept is that genes encoding synthetic signaling components can be introduced into cells and the encoded proteins can query whether a specific oncogenic signal exists. If this system can differentiate constitutive oncogenic signals from normal transient signals, then the therapeutic program could be specifically triggered only in cancer cells, preventing undesired toxicities in normal tissues.

For rewiring of endogenous oncogenic signals to therapeutic outputs to become a feasible approach, the components should be compatible with delivery by known non-toxic gene-expression vectors. We thus aimed for our system to be encodable within the 4.7-kilobase packaging limit of adeno-associated virus, a non-integrating virus with a strong clinical safety record. For therapeutic versatility, the ability to program the system to produce multiple specific outputs would be highly desirable as well. These conditions suggested that a system that can activate selected endogenous genes would be ideal, as useful therapeutic functions can be found in the library of 20,000 genes in the human genome.

Several natural and engineered systems demonstrate that two-component systems can link the presence of a molecular signal to transcription with high responsivity. In bacterial two-component systems, ligand binding to a receptor kinase induces it to phosphorylate a cytosolic transduction protein, inducing its latent transcription factor activity. The Notch receptor protein responds to presentation of the ligand Delta on contacting cell surfaces by undergoing presenilin-mediated transmembrane cleavage, allowing a polypeptide fragment to translocate to the nucleus to induce gene transcription. Presenilin-mediated cleavage has been found to occur even when Notch and Delta extracellular domains are swapped for other protein-protein interactions, allowing transcriptional programs to be linked to specific cell-cell contacts. In the synthetic TANGO system, two synthetic proteins are expressed to detect GPCR ligands. Ligand induces binding between one protein, a fusion of G-protein-coupled receptor (GPCR) and tobacco etch virus (TEV) protease, to the other protein, a fusion of beta-arrestin, TEV substrate, and a transcription factor, leading to transcription factor release. However, while the above synthetic systems exist to link transmembrane or extracellular ligands to gene transcription, simple synthetic systems for linking intracellular oncogenic signals to therapeutic outputs do not exist. Such a system would need to solve three challenges: First, the presence of an endogenous oncogenic signal would need to be converted to a therapeutic output, and second, oncogenic levels of the signal would need to be differentiated from normal patterns of signal activation.

In this study, we describe the engineering and application of a compact two-component system to senses constitutively ErbB phosphorylation and triggers therapeutic responses. We have created a system for detecting hyperactive signaling from ErbB receptor tyrosine kinases, which occurs in a large fraction of solid tumors, especially breast, colorectal, head and neck, brain, and lung cancers. This system comprises two proteins, one of which contains a viral protease domain and is expressed as a cytosolic protein, and the other which consists of a therapeutic cargo protein that is linked to a membrane-targeting sequence via a substrate sequence for the cytosolic viral protease. Both proteins are recruited to active ErbB receptor intracellular domains by phosphotyrosine-binding domains so that protease induces release of cargo from the membrane tether in proportion to ErbB signal duration. The use of a modular architecture facilitates customization of inputs and outputs and optimization of the system as a whole. Mathematical modeling of the entire system enables in silico optimization of several biochemical parameters to further enhance system responsivity. The resulting system for ErbB-specific rewiring of aberrant signaling to effector release (ErbB-RASER) responds specifically to constitutively active ErbB, is as sensitive to constitutive ErbB signaling as native growth- and survival-promoting kinase pathways, and can be programmed to induce a variety of outputs including direct induction of apoptosis and transcription of apoptosis-inducing genes.

Results

The Concept of a Synthetic Two-Component System Based on Signaling Dependent Proteolysis

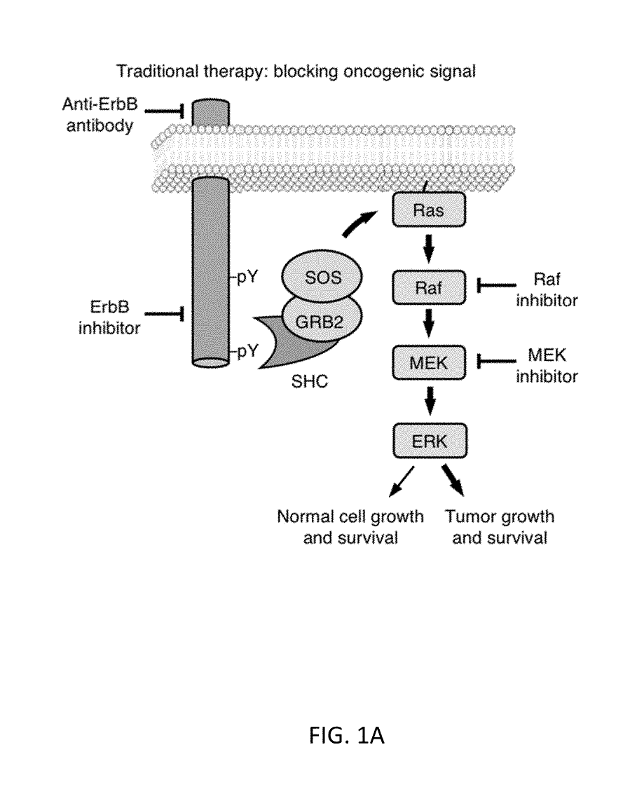

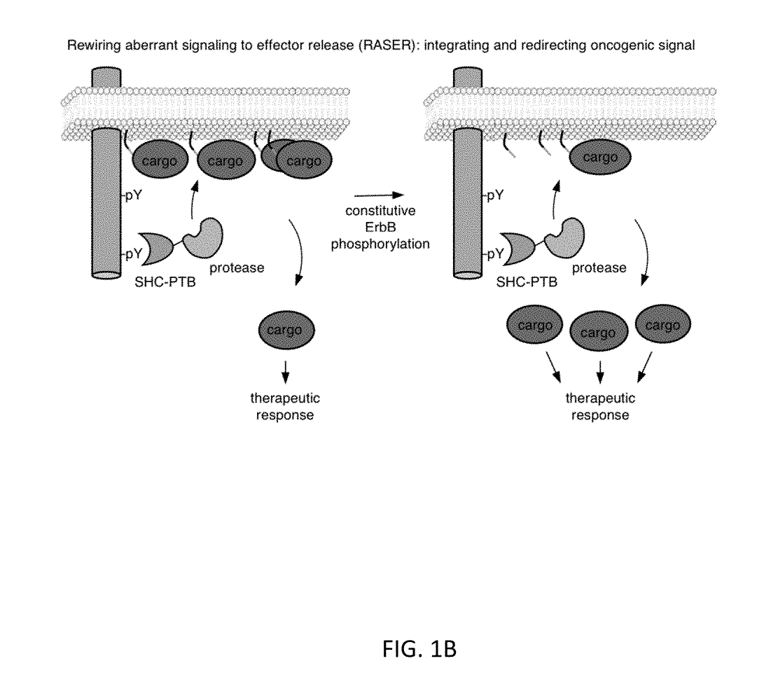

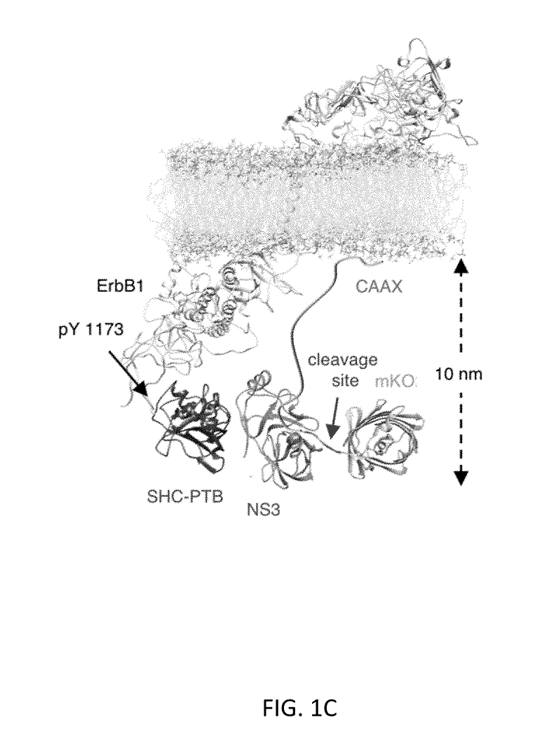

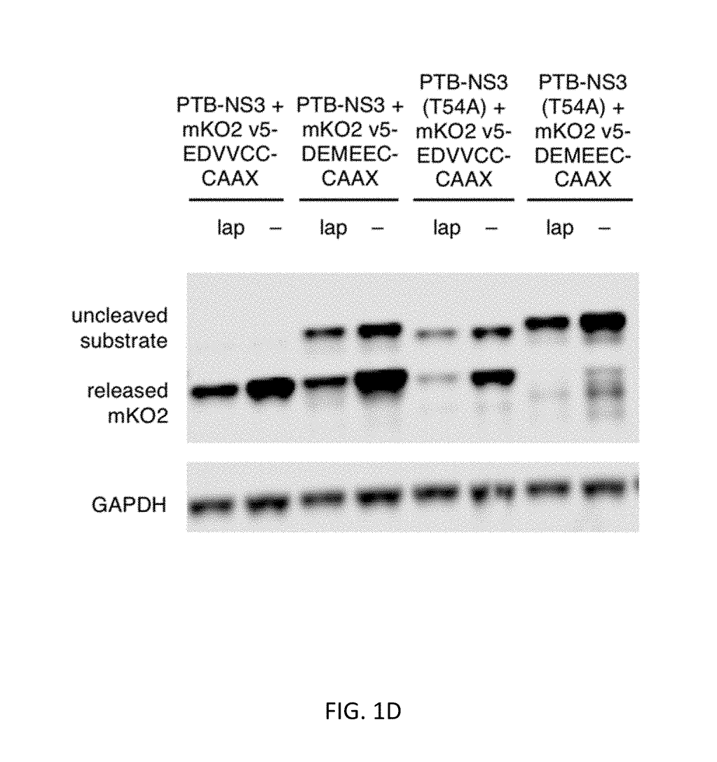

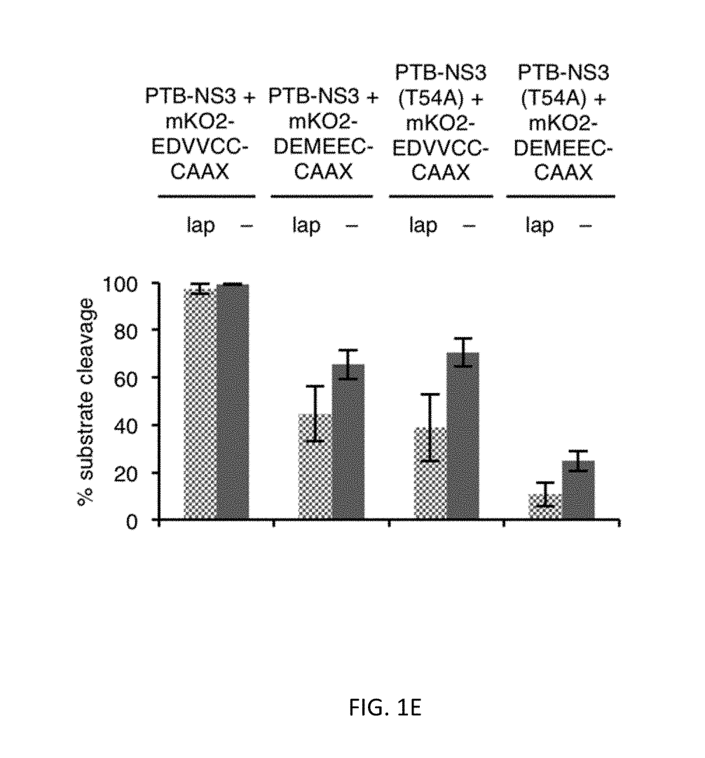

To specifically sense a cancer state, we considered how to detect the difference between physiological signaling, which is transient, with oncogenic signaling, which is constitutive. To link signaling to various outputs, we considered how to activate a variety of different effectors using a common mechanism. We conceived the idea of using signal-induced proteolysis as a mechanism for integrating signal activity over time, and as a generalizable activation mechanism for multiple effectors (FIG. 1B). Specifically, because proteolysis is irreversible, the products can accumulate proportionally to protease activity integrated over time. Secondly, many effector domains can be functionally inactivated by appending a motif that localizes the effector away from a required site of function, which can then be reversed by proteolytic removal of the localization motif. We termed this general approach of linking cargo release to oncogenic signaling via a two-component protease-substrate system as Rewiring of Aberrant Signaling to Effector Release (RASER).

As a first system, we aimed to detect the ErbB-family of receptor tyrosine kinases (RTKs), which include ErbB1 (HER1, EGFR) and ErbB2 (HER2, Neu) which are constitutively phosphorylated in 30% of solid tumors. Oncogenic mutations or overexpression of ErbB leads to its constitutive phosphorylation at cytoplasmic tyrosine residues, which then bind to phosphotyrosine-binding (PTB) and SH2 domains. Domains that bind to active ErbB proteins have been extensively characterized, including measurements of binding affinities in high-throughput experiments. Furthermore, as the site of corecruitment will be the membrane, the substrate-effector fusion, can be prelocalized to the membrane. This should sequester the substrate away from the majority of cytosolic protease molecules, reducing basal cleavage rates. To then achieve proteolysis in a manner dependent on the integrated ErbB signal over time, we postulated we could bring a weak protease to the membrane in a signal-dependent manner by attaching a phosphotyrosine binding (PTB) domain that can bind to active ErbB receptor. The binding of the fusion proteins to the oncogenic signal should effectively concentrate the substrate in the vicinity of the protease, allowing for higher enzyme occupancy by substrate and thereby faster effector release.

To test this concept, we first constructed a simple system. We considered which domain to fuse to HCV NS3 protease to recruit it to the membrane in an ErbB phosphorylation-dependent manner. High affinity should maximize receptor occupancy, so we selected Shc PTB as the targeting domain for the protease, as it has the highest known affinity for phosphorylated ErbB RTKs. We localized substrate to the membrane via a CAAX farnesylation signal and used the orange fluorescent protein mKO2 as a mock effector. Molecular modeling suggested the mKO2-substrate-CAAX protein should be able to be cleaved by ShcPTB-NS3 bound to ErbB (FIG. 1C). We tested combinations of two HCV protease variants and two substrate variants in BT-474 breast cancer cells, which overexpress ErbB2. For a matched ErbB-inactive control, we treated the same cells with the ErbB kinase inhibitor lapatinib. We observed a range of cleavage efficiencies, with nearly complete cleavage with medium-speed protease and high-affinity substrate (FIGS. 1D and 1E). These results thus also allow us to rule out TEV protease, which exhibits even faster cleavage of its substrate. However, they also showed that a simple system with only protease recruitment to the receptor is insufficient for inducible effector release from a farnesyl membrane anchor. Specifically, the maximum fold induction observed (approximately 2.5-fold) was with the slower-cleaving T54A mutant of NS3 protease, but cleavage efficiency was low, at only 25% after 24 hours. Thus, a system comprising a PTB-protease fusion and membrane-bound substrate did not demonstrate robust ErbB-dependent effector release.

Dual Targeting of RASER Components Improves Responsivity

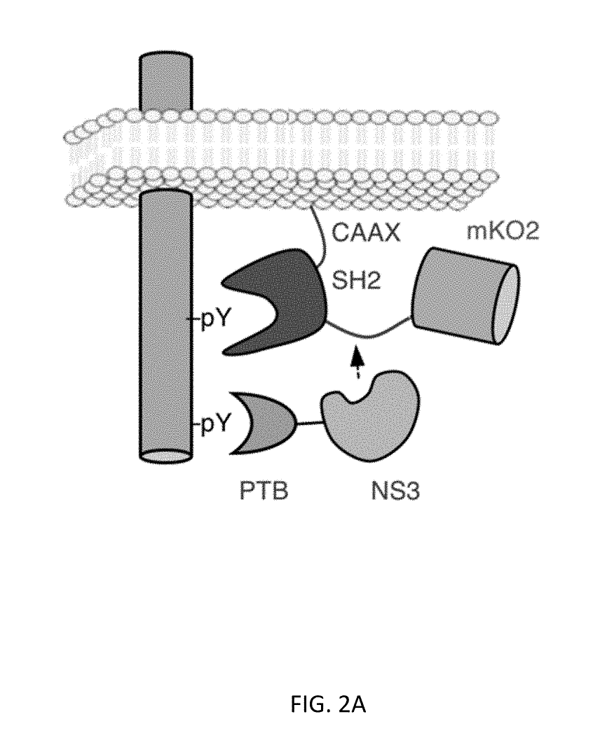

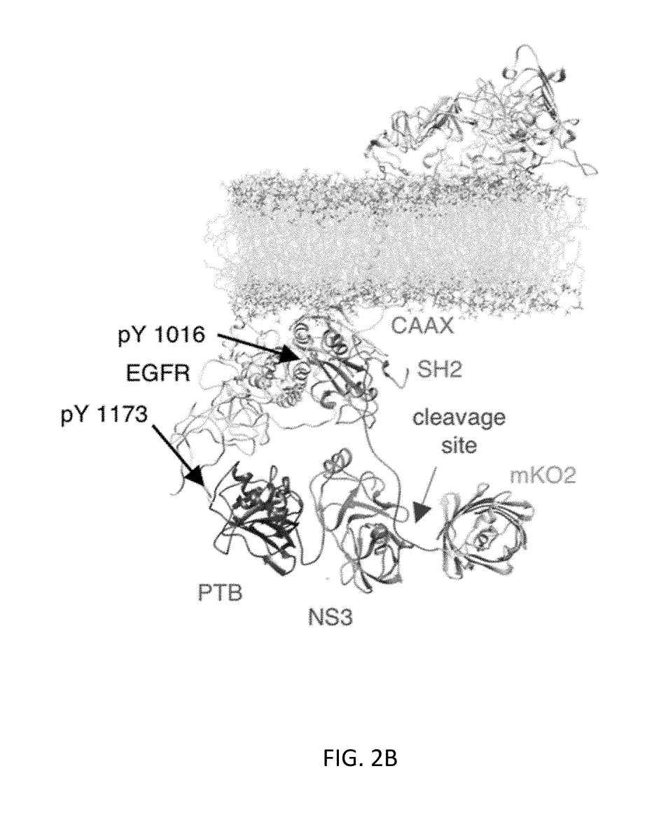

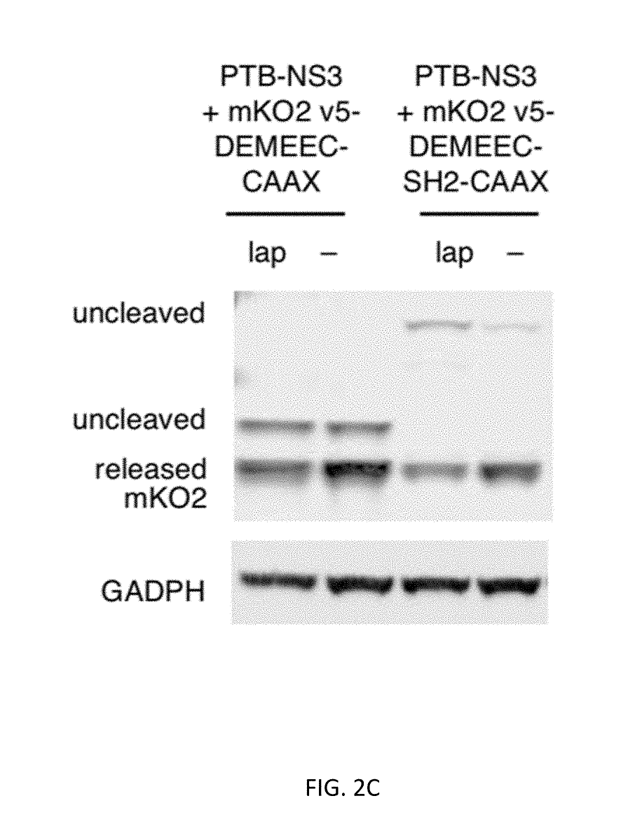

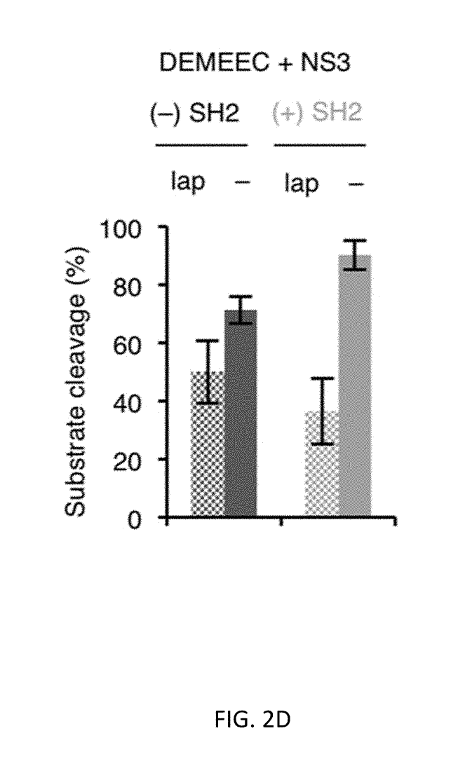

To improve dynamic range, we explored the possibility of binding protease and substrate simultaneously to active ErbB receptors (FIG. 2A). Like PTB, fused to the protease, the other domain is fused to the substrate and the effector. The binding of both fusion proteins to the same oncogenic signal will concentrate protease to the site of substrate, increasing the total number of proteases engaged with substrate. To achieve accumulation of effector, we note that the substrate-effector fusion needs to be capable of rapid dissociation, and substrate needs to be in excess over protease.

We first used structural modeling to select SH2 domains targeting active ErbB for the substrate that do not interfere with PTB-protease binding. In ErbB1, Tyr1016 is close enough to allow protein binding there to be cleaved by a ShcPTB-protease fusion binding at Tyr1173 (FIG. 2B), yet does not confer steric hindrances between the protease and the substrate components. When substrate was targeted to active receptor using a Vav1 SH2 domain, cleavage was robustly dependent on constitutive ErbB activity (FIGS. 2C and 2D).

Destabilizing Protease to Further Improves Responsivity

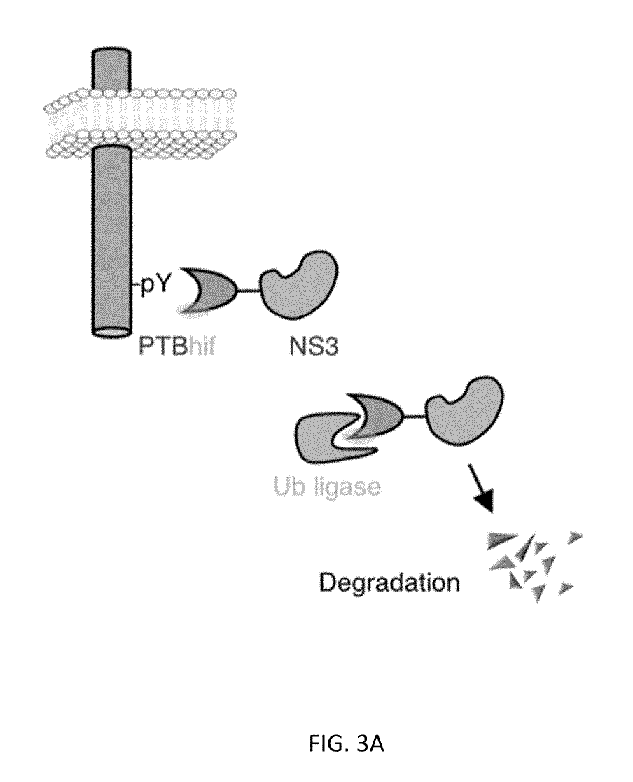

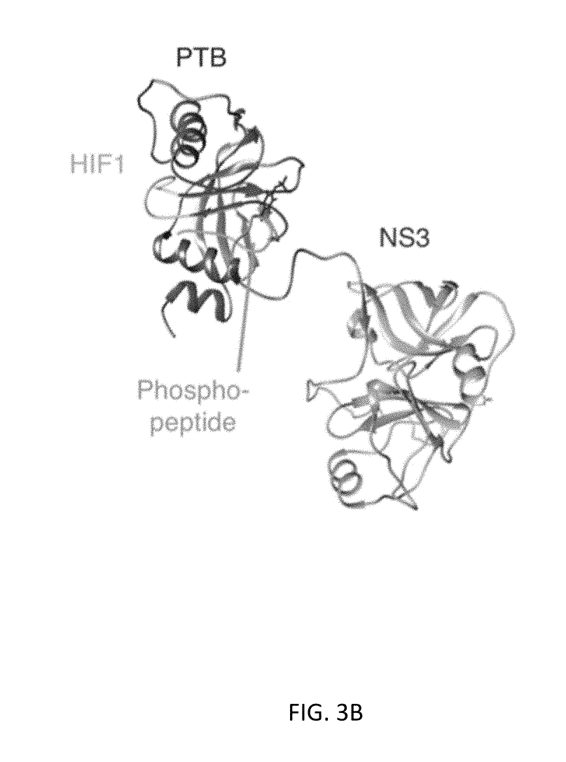

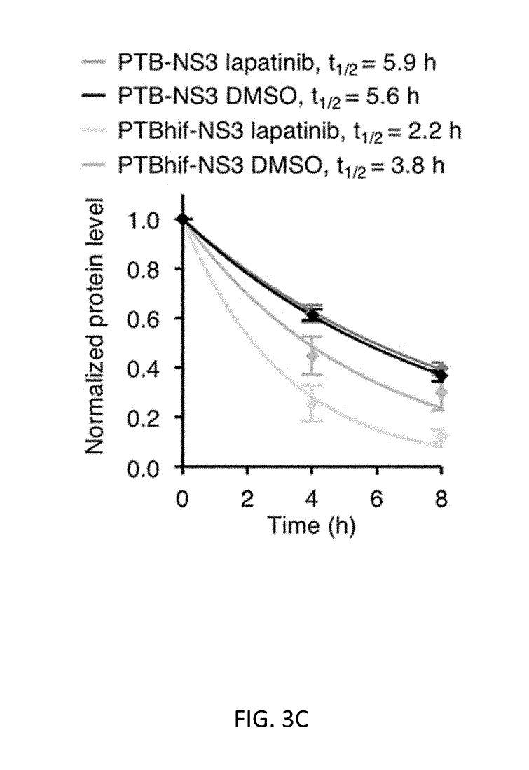

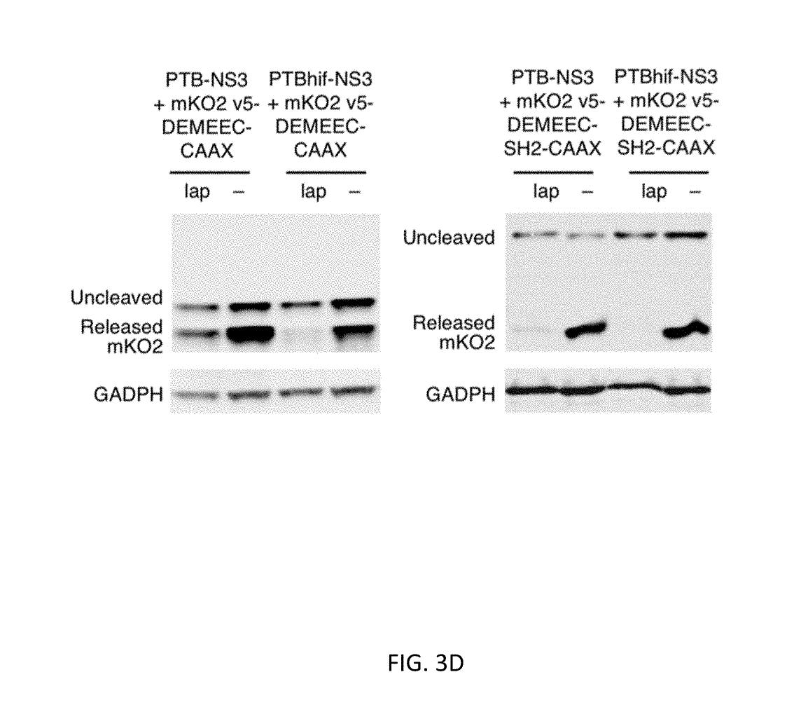

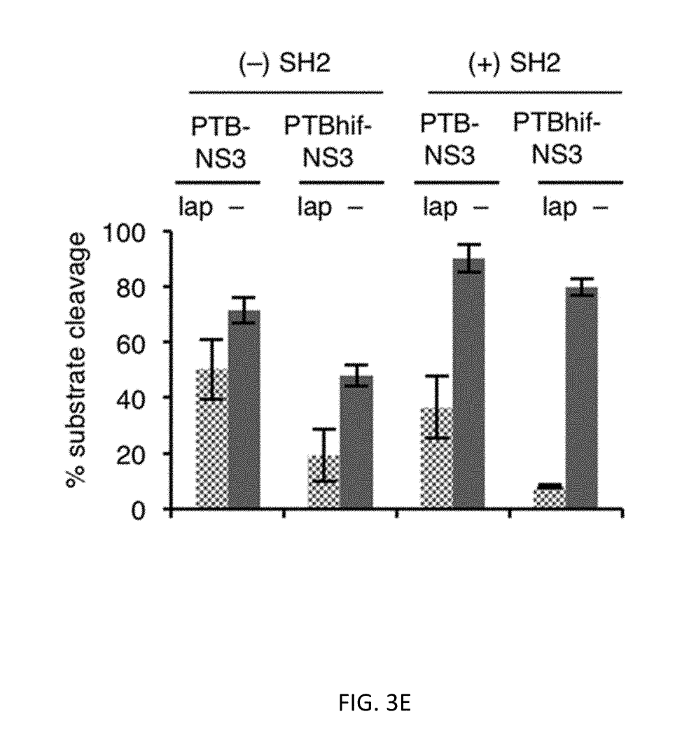

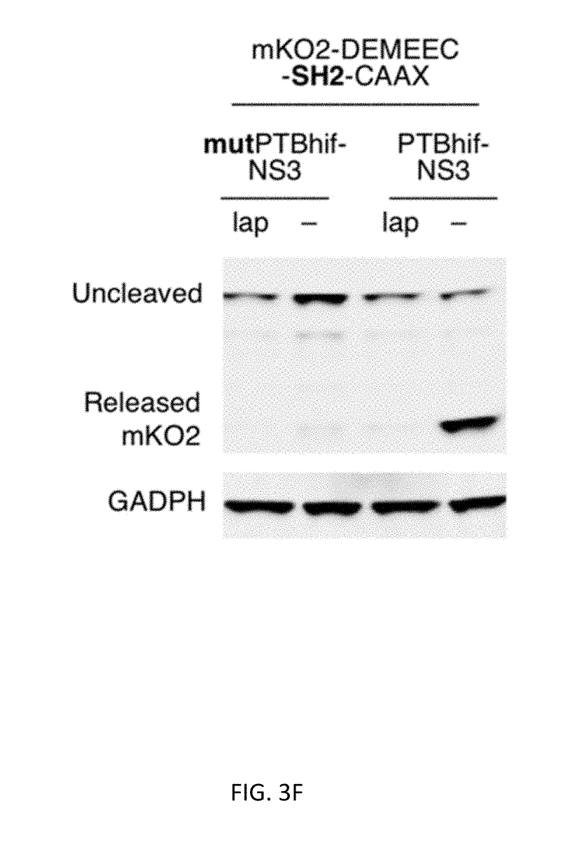

How can we further suppress activity in the ErbB-off state? In addition to using receptor to localize protease to the membrane, we conceived an idea of using the receptor to stabilize the protease. We hypothesized we could attach a degron to the protease whose function would be blocked by receptor binding (FIG. 3A). This would have the beneficial effect of allowing protease to accumulate preferentially in cancer cells, where it will induce more effector release. Using molecular modeling, we placed a short peptide degron from HIF1a in a loop of the PTB domain near the phosphopeptide-binding groove (FIG. 3B). Our empirical experiments confirmed a shorter half-life in the absence of the phosphorylated receptor than in the presence of the active receptor (FIG. 3C). Substrate cleavage in the absence of ErbB activity was reduced, increasing the fold induction of substrate release in ErbB-overexpressing cancer cells (FIGS. 3D and 3E). The best performance was observed with PTBhif-NS3 and cargo-DEMEEC-SH2-CAAX, so these two proteins was designated as the ErbB-RASER system. Finally, we confirmed that performance of ErbB-RASER depends on both PTB and SH2 targeting (FIG. 3F).

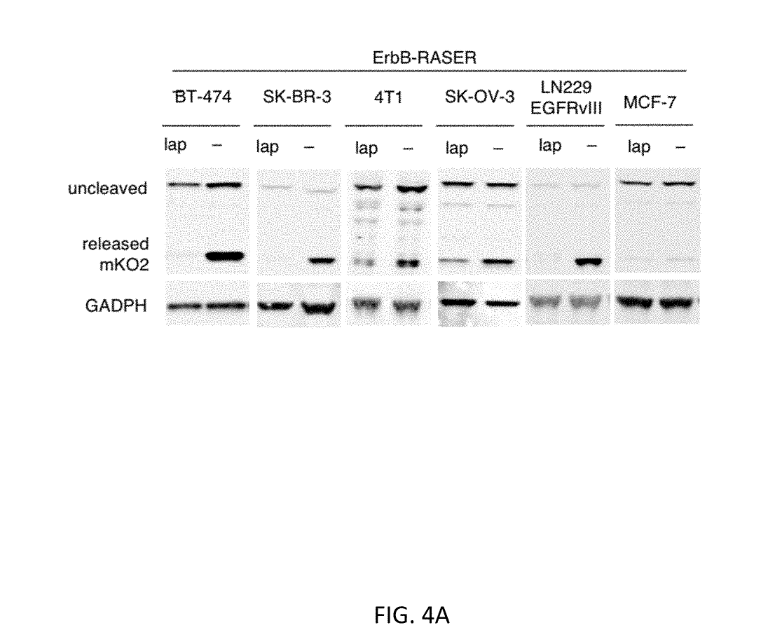

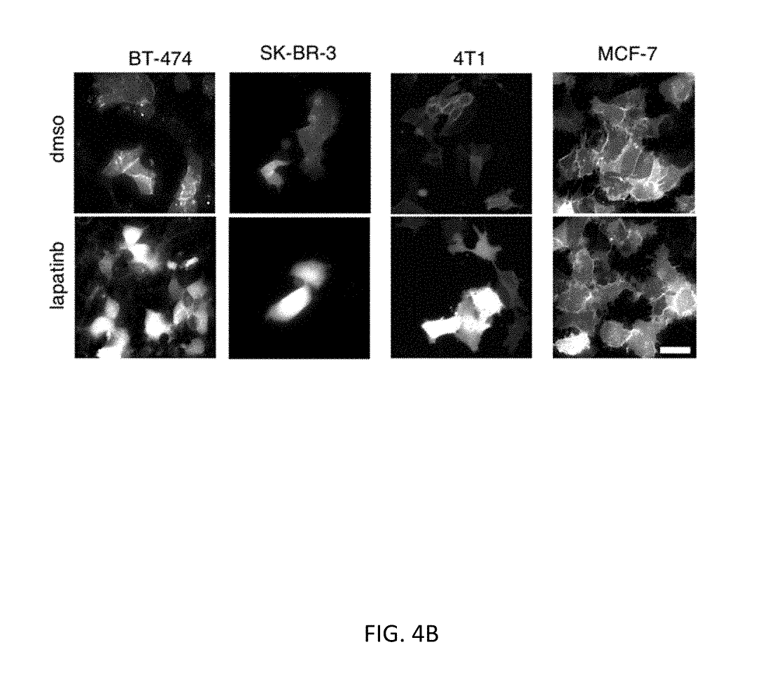

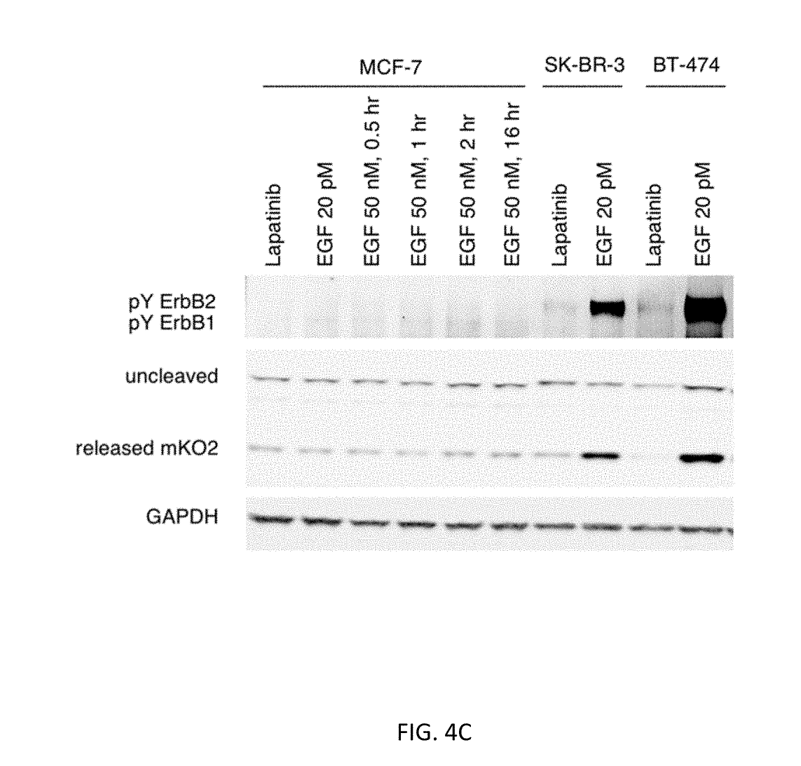

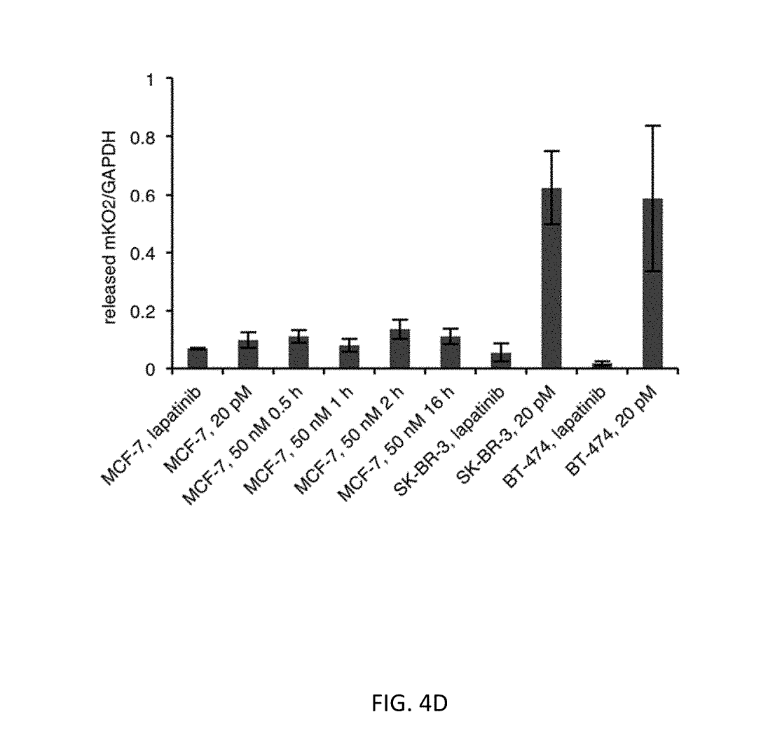

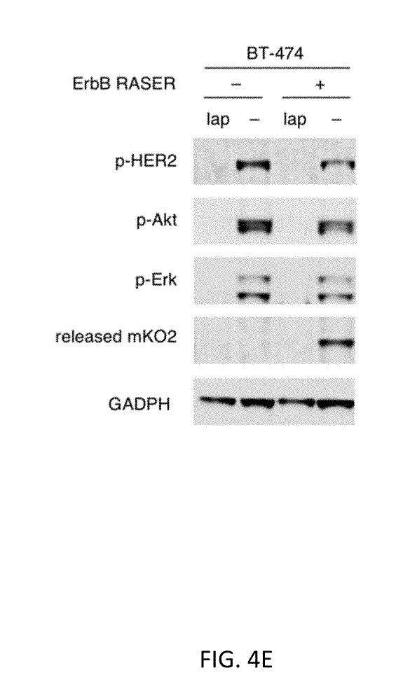

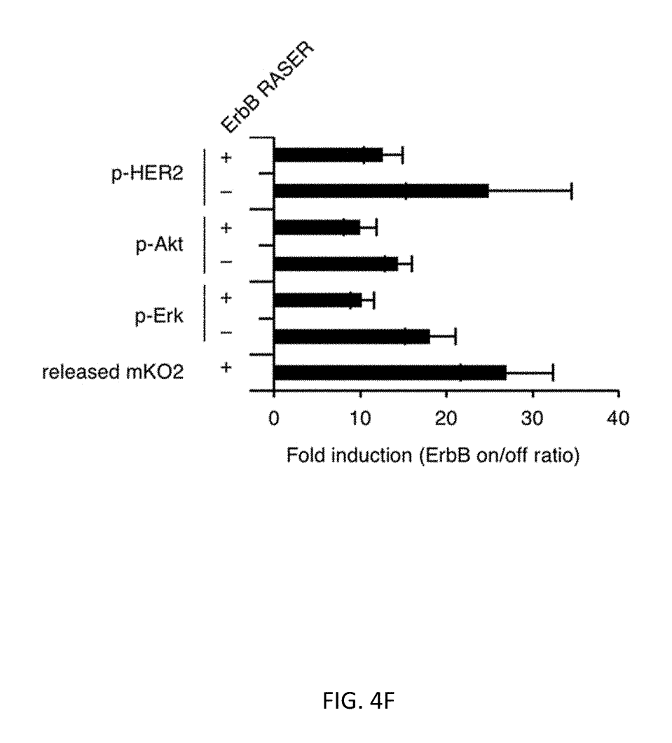

We tested the specificity and inducibility of ErbB-RASER in various cancer cell lines. To test the generalizability of ErbB-RASER for cells with hyperactive ErbB, we tested substrate cleavage in various cancer cell lines known to overexpress ErbB, including glioblastoma, breast cancer, and ovarian cancer cells. ErbB-RASER expression resulted in cargo release in an ErbB-dependent manner all ErbB-overexpressing lines, but not in MCF-7 breast cancer cells, which express normal levels of ErbB receptors (FIGS. 4A and 4B). To test the specificity of ErbB-RASER for cells with constitutively active ErbB, we tested whether EGF stimulation of MCF-7 cells could induce ErbB-RASER cargo release. ErbB1 in MCF-7 is phosphorylated upon EGF stimulation while ErbB2 in SK-BR-3 and BT-474 is constitutively phosphorylated (FIGS. 4C and 4D). We found that cargo release in MCF-7 remains low even after EGF stimulation, whereas cargo release is high in SK-BR-3 and BT-474 cells even without EGF. Thus, as intended, RASER selectively responds to aberrant cancer signaling rather than normal ErbB activation.

Interestingly, the complete RASER system showed a similar or larger degree of dependence on ErbB activation than endogenous signaling pathways. For example, mKO2 release increased 27-fold in BT-474 cells between ErbB-inhibited and ErbB-active states, whereas endogenous Akt and Erk phosphorylation levels increased only 14- and 18-fold (FIGS. 4E and 4F).

Programming of RASER with a Variety of Outputs

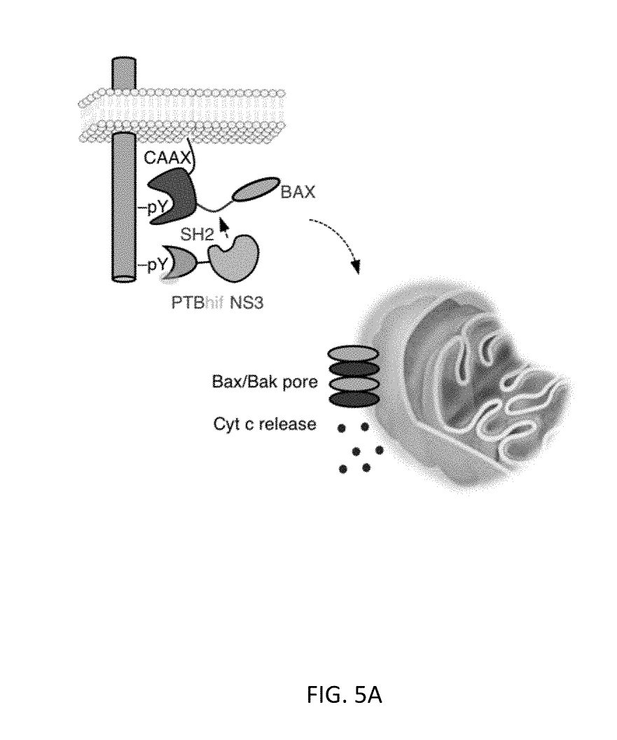

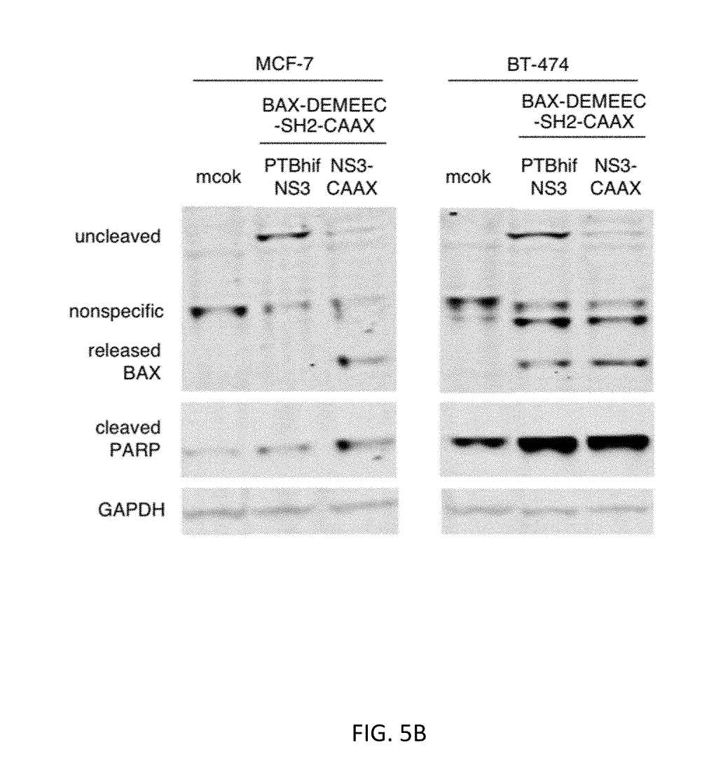

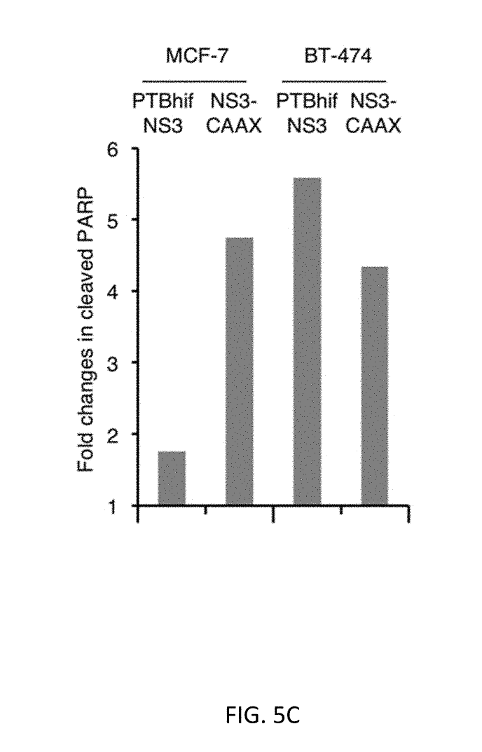

Now that we have built a synthetic signaling system that has the unique ability of integrating signal from ErbB over time and thus specifically detecting oncogenic ErbB, we explored different options for programmable cargos. Cargos causing cell death could be therapeutically useful to release in ErbB-hyperactive cancer cells. To test this, we created an ErbB-RASER system in which the cargo protein is Bax, a protein that induces cytochrome release from mitochondria to initiate apoptosis (FIG. 5A). We then tested this ErbB-RASER-Bax system in BT-474 cells which overexpress ErbB receptors and MCF-7 cells with normal ErbB levels (FIG. 5B). We found that ErbB-RASER-Bax was indeed able to induce apoptosis in BT-474 cells in an ErbB signaling-dependent manner, with levels of the apoptotic marker PARP reaching similar levels as with direct membrane expression of protease (FIGS. 5B and 5C). In contrast, in MCF-7 cells, PARP levels remain near untransfected controls (FIGS. 5B and 5C). These results establish that the RASER system can be designed to trigger a therapeutic function in response to an oncogenic state.

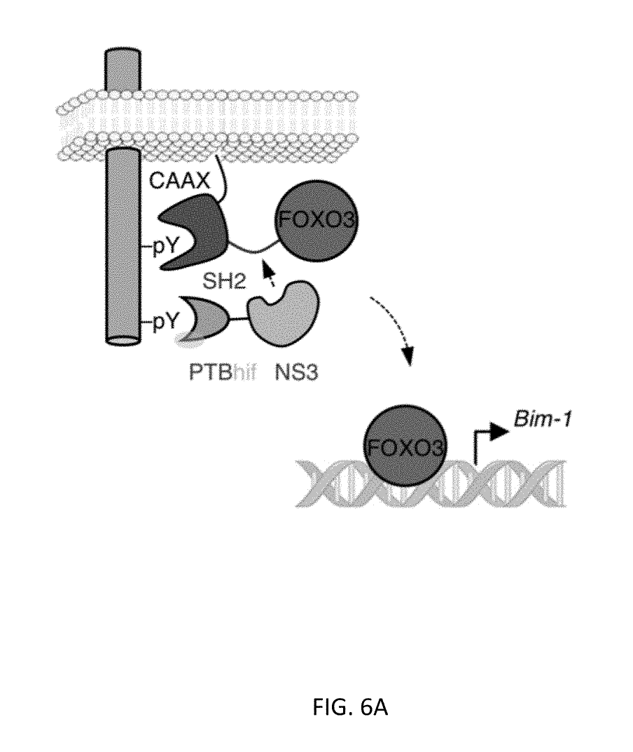

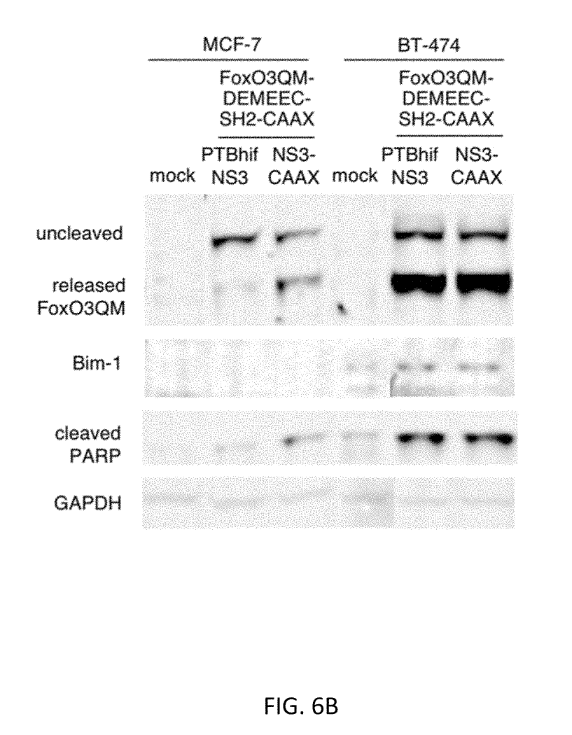

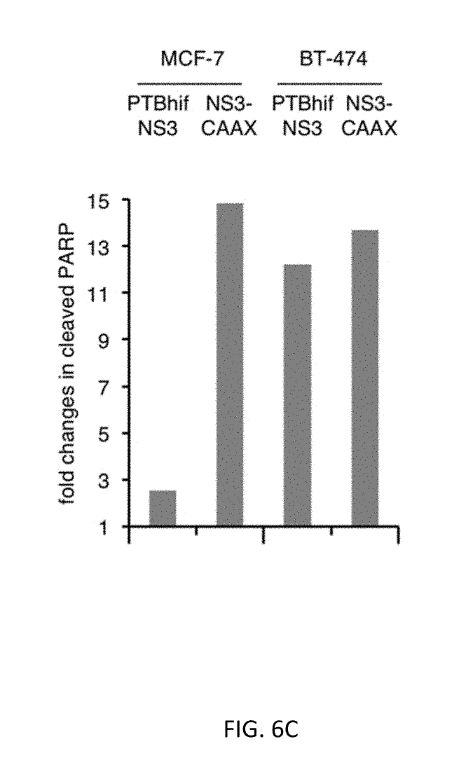

Another general class of useful cargos for ErbB-RASER may be transcription factors that can activate endogenous genes in cancer cells for therapeutic effect. We thus also created an ErbB-RASER system in which the cargo protein is a constitutively active FoxO3, a transcription factor that activates pro-apoptotic genes (FIG. 6A). We also tested this ErbB-RASER-FoxO system in BT-474 cells which overexpress ErbB receptors and MCF-7 cells with normal ErbB levels (FIG. 6B). We found that ErbB-RASER-FoxO was also able to induce apoptosis in BT-474 cells in an ErbB signaling-dependent manner, with levels of the apoptotic marker PARP reaching similar levels as with direct membrane expression of protease (FIGS. 6B and 6C). In contrast, in MCF-7 cells, PARP levels remains near untransfected controls (FIGS. 6B and 6C). These results establish that the RASER system can be designed to trigger a therapeutic function in response to an oncogenic state via activation of endogenous genes.

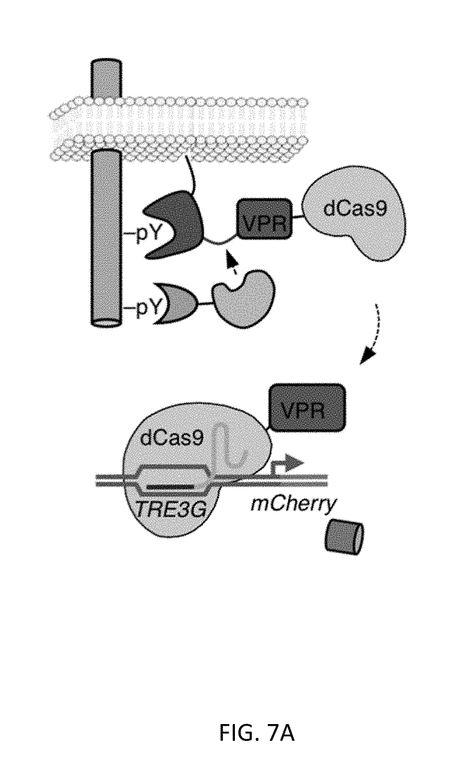

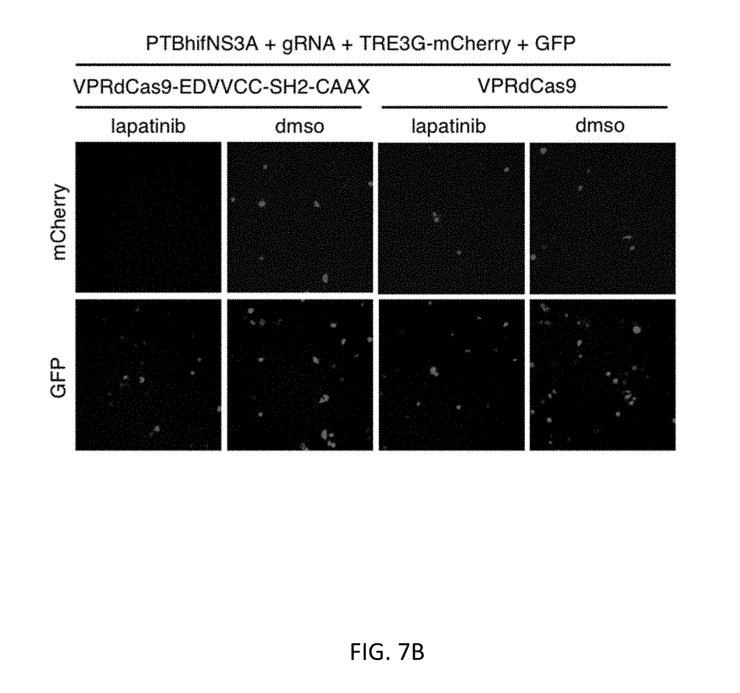

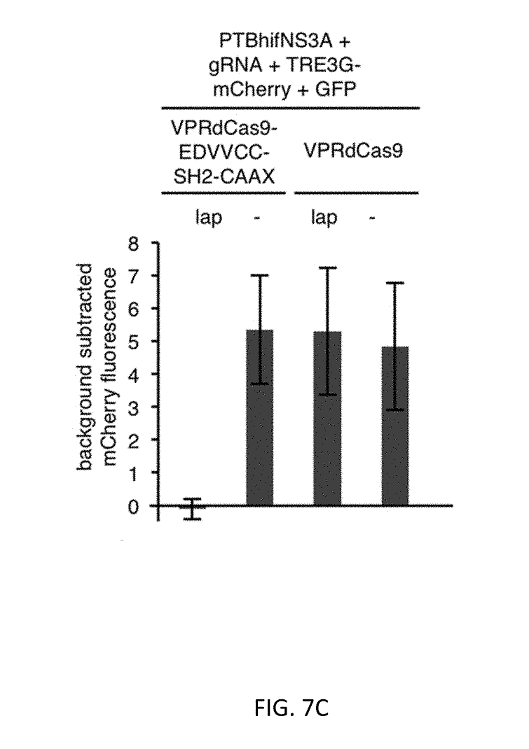

Finally, we explored whether RASER could be used to rewire hyperactive ErbB to the transcriptional activation of essentially any endogenous gene by using a CRISPR/Cas9 protein as the cargo. Catalytically inactive Cas9 (dCas9) fused to the VP64-p65-Rta-dCas9 (VPR) transcriptional activation domain can be targeted by a coexpressed guide RNA (gRNA) to promote transcription of a gene of interest. We generated an ErbB-RASER-VPRdCas9 system to release VPRdCas9 in an ErbB-dependent manner (FIG. 7A). To test ErbB-RASER-VPRdCas9, we expressed in BT474 cells the RASER components, a reporter plasmid that expresses a mCherry gene under the control of a TRE promoter, and a gRNA targeting the TRE promoter. Cells were then left untreated or treated with lapatinib to shut off the ErbB signal. Indeed, we observed that, in the absence of lapatinib, RASER VPR-dCas9 induces mCherry expression as well as the parent VPRdCas9 (FIGS. 7B and 7C). Lapatinib prevents mCherry expression in RASER-transfected cells, but not in cells expressing a positive control VPRdCas9 construct and gRNA, demonstrating the requirement for ErbB signaling (FIGS. 7B and 7C). These results establish that the RASER system can be programmed to induce dCas9-mediated activation of a promoter specified by a coexpressed gRNA.

Discussion

To summarize, we have provided proof of concept for a new approach called RASER in which we construct an artificial signaling pathway to rewire oncogenic signaling states to effector activation. Importantly, this synthetic signaling pathway is compact, comprising only two proteins, and can be programmed to activate a variety of outputs. For example, we have found RASER can be programmed to release BAX to activate an endogenous apoptotic pathway, to release FoxO to activate endogenous transcription, and to release VPRdCas9 to activate genes targeted by a gRNA. We believe that this programmability will be broadly useful, as it will allow ErbB hyperactivity to be rewired to a variety of therapeutically useful outputs, such as induction of apoptosis or activation of immunostimulatory genes.

As a therapeutic approach, RASER may be advantageous over conventional therapies in that it is unlikely to elicit drug resistant mutations. Conventional therapies such as RTK inhibitors and monoclonal antibodies inhibit cell proliferation via inhibiting kinase activity or binding to the ectodomain of the receptor providing a strong selective pressure for target mutations that mitigate inhibitor binding and preserve receptor function. In contrast, RASER is activated by the same signals used by the cell to drive tumor growth and survival. Thus, further increases in ErbB activity should only activate RASER further, whereas mutations that decrease RASER activation, such as at phosphoacceptor sites in ErbB, would result in loss of oncogenic drive as well.

Methods

DNA Constructs.

Plasmids encoding RASER cassettes were cloned by standard molecular biology techniques including PCR, restriction enzyme digestion and ligation or In-Fusion enzyme (Clontech). All subcloned fragments were sequenced in their entirety to confirm successful construction. Full sequences of all plasmids used in this study are available upon request.

Cell Culture and Transfection.

BT-474 (ATCC), SK-BR-3(ATCC), 4T-1 (gift from Dr. Ronald Levy at Stanford University) cell lines were cultured at 37° C. in 5% CO₂ in RPMI 1640 medium (Life Technoloiges) supplemented with 10% FBS (Gibco), and 100 U/mL penicillin and 100 μg/mL streptomycin (Life Technologies). MCF-7 (gift from Dr. Howard Chang at Stanford University), SK-OV-3 (gift from Dr. Hongjie Dai at Stanford University), and LN-229 EGFRvIII (gift from Xiaokun Shu at UCSF) cell lines were cultured at 37° C. in 5% CO₂ in Dulbecco's Modified Eagle's Medium (DMEM, HyClone) supplemented with 10% FBS (Gibco) and 100 U/mL penicillin and 100 μg/mL streptomycin (Life Technologies). Cells were transfected using Lipofectamine 3000 (Life Technologies) in Opti-MEM (Life Technologies) according to the manufacturer's recommended protocol.

Microscopy.

Fluorescence imaging was performed on a Zeiss Axiovert 200M with a 10×/0.25-numerical aperture (NA) objective. Cells were cultured in 12-well plates (Greiner) and imaged in culture media. The microscope was connected to Hamamatsu ORCA-ER cameras and controlled by Micro-Manager software. Image processing was performed in ImageJ.

Immunoblotting.

After washing twice with PBS, cells were lysed with 50-100 μl of hot SDS lysis buffer (100 mM Tris HCl pH 8.0, 4% SDS, 20% glycerol, 0.2% bromo-phenol blue, 10% 2-mercaptoethanol) and DNA was sheared by sonication. After heating to 80-90° C. for several minutes, cell lysates were loaded onto 4%-12% Bis-Tris gels (NuPAGE, Life Technologies) along with Novex Sharp pre-stained protein standard (Life Technologies) or Precision Plus Protein Dual Color Standards (Bio-Rad). Gels were transferred to nitrocellulose membranes using Trans-Blot Turbo Transfer System (Bio-Rad). Membranes were probed with primary and secondary antibodies, and imaged using LI-COR Odyssey imaging system. Quantification of immunoblots was performed in ImageJ.

Apoptosis Assay.

After washing twice with PBS, cells were lysed with 50-100 μl of hot SDS lysis buffer (100 mM Tris HCl pH 8.0, 4% SDS, 20% glycerol, 0.2% bromo-phenol blue, 10% 2-mercaptoethanol) and DNA was sheared by sonication. After heating to 80-90° C. for several minutes, cell lysates were loaded onto 4%-12% Bis-Tris gels (NuPAGE, Life Technologies) along with Novex Sharp pre-stained protein standard (Life Technologies) or Precision Plus Protein Dual Color Standards (Bio-Rad). Gels were transferred to nitrocellulose membranes using Trans-Blot Turbo Transfer System (Bio-Rad). Membranes were probed with primary and secondary antibodies, and imaged using LI-COR Odyssey imaging system. Quantification of immunoblots was performed in ImageJ.

While the preferred embodiments of the invention have been illustrated and described, it will be appreciated that various changes can be made therein without departing from the spirit and scope of the invention.

What is claimed is:

1. A method for targeted treatment of a cancer associated with hyperactivity of a receptor tyrosine kinase, the method comprising:
a) administering to a subject in need thereof a therapeutically effective amount of a first fusion protein comprising a protease connected to a phosphotyrosine binding (PTB) domain capable of binding to a phosphorylated tyrosine residue on the receptor tyrosine kinase; and
b) administering a therapeutically effective amount of a second fusion protein comprising an SH2 domain connected to i) a substrate comprising a cleavage site recognized by the protease and ii) an anti-cancer therapeutic agent, wherein cleavage of the substrate at the cleavage site by the protease of the first fusion protein releases the anti-cancer therapeutic agent from the second fusion protein.

2. The method of claim 1, wherein the receptor tyrosine kinase is a hyperactive ErbB receptor tyrosine kinase.

3. The method of claim 1, wherein the protease is a hepatitis C virus (HCV) NS3 protease.

4. The method of claim 1, wherein the first fusion protein further comprises a degron, wherein degradation activity of the degron is inhibited by binding of the PTB domain of the fusion protein to the phosphorylated tyrosine residue on the receptor tyrosine kinase such that the fusion protein accumulates preferentially in cancerous cells.

5. The method of claim 4, wherein the degron is located in a loop of the PTB domain.

6. The method of claim 4, wherein the degron is a HIF1a degron.

7. The method of claim 1, wherein the PTB is a Shc PTB.

8. The method of claim 1, wherein the SH2 domain is a Vav1 SH2 domain.

9. The method of claim 1, wherein the tyrosine kinase receptor is constitutively phosphorylated at the tyrosine residue.

10. The method of claim 1, wherein the cancer is selected from the group consisting of breast cancer, colorectal cancer, head and neck cancer, brain cancer, and lung cancer.

11. The method of claim 1, wherein the first fusion protein or the second fusion protein is provided by a vector.

12. The method of claim 12, wherein the vector is a non-viral or viral vector.

13. The method of claim 13, wherein the viral vector is a non-integrating viral vector.

14. The method of claim 1, wherein the anti-cancer therapeutic agent is a pro-apoptotic protein or a transcription factor that activates a pro-apoptotic gene.

15. The method of claim 14, wherein the pro-apoptotic protein is BAX.

16. The method of claim 14, wherein the transcription factor is FoxO3.

17. The method of claim 1, wherein the anti-cancer therapeutic agent comprises a complex of a catalytically inactive Cas9 (dCas9) with a guide RNA for activating or repressing expression of a gene of interest.

18. The method of claim 17, wherein the dCas9) is fused to a transcriptional activation domain capable of activating transcription of a gene of interest.

19. The method of claim 18, wherein the gene of interest is a pro-apoptotic gene or an immunostimulatory gene.

20. The method of claim 18, wherein the transcriptional activation domain is a VP64-p65-Rta (VPR) transcriptional activation domain.

21. The method of claim 1, wherein multiple cycles of treatment are administered to the subject for a time period sufficient to effect at least a partial tumor response.

22. The method of claim 21, wherein multiple cycles of treatment are administered to the subject for a time period sufficient to effect a complete tumor response.

23. A method of selectively treating a cancerous cell having a hyperactive ErbB receptor tyrosine kinase in a heterogenous population of cells, the method comprising:
a) contacting the population of cells with an effective amount of a first fusion protein comprising a protease connected to a phosphotyrosine binding (PTB) domain that selectively binds to a phosphorylated tyrosine residue on the hyperactive receptor tyrosine kinase; and
b) contacting the population of cells with an effective amount of a second fusion protein comprising an SH2 domain connected to i) a substrate comprising a cleavage site recognized by the protease and ii) an anti-cancer therapeutic agent, wherein cleavage of the substrate at the cleavage site by the protease of the first fusion protein releases the therapeutic agent from the second fusion protein inside the cancerous cell having the hyperactive ErbB receptor tyrosine kinase.

24. The method of claim 23, wherein the protease is a hepatitis C virus (HCV) NS3 protease.

25. The method of claim 23, wherein the first fusion protein further comprises a degron, wherein degradation activity of the degron is inhibited by binding of the PTB domain of the fusion protein to the phosphorylated tyrosine residue on the receptor tyrosine kinase such that the fusion protein accumulates preferentially in cancerous cells.

26. The method of claim 25, wherein the degron is located in a loop of the PTB domain.

27. The method of claim 25, wherein the degron is an HIF1a degron.

28. The method of claim 23, wherein the PTB is a Shc PTB.

29. The method of claim 23, wherein the SH2 domain is a Vav1 SH2 domain.

30. The method of claim 23, wherein the tyrosine kinase receptor is constitutively phosphorylated at the tyrosine residue.

31. The method of claim 23, wherein the first fusion protein or the second fusion protein is provided by a vector.

32. The method of claim 31, wherein the vector is a non-viral or viral vector.

33. The method of claim 32, wherein the viral vector is a non-integrating viral vector.

34. The method of claim 23, wherein the anti-cancer therapeutic agent is a pro-apoptotic protein or a transcription factor that activates a pro-apoptotic gene.

35. The method of claim 34, wherein the pro-apoptotic protein is BAX.

36. The method of claim 34, wherein the transcription factor is FoxO3.

37. The method of claim 23, wherein the anti-cancer therapeutic agent comprises a complex of a catalytically inactive Cas9 (dCas9) with a guide RNA for activating or repressing expression of a gene of interest.

38. The method of claim 37, wherein the dCas9) is fused to a transcriptional activation domain capable of activating transcription of a gene of interest.

39. The method of claim 38, wherein the gene of interest is a pro-apoptotic gene or an immunostimulatory gene.

40. The method of claim 38, wherein the transcriptional activation domain is a VP64-p65-Rta (VPR) transcriptional activation domain.
